(19) United States
(12) Patent Application Publication
(10) Pub. No.: US 2003/0105308 A1
(43) Pub. Date: Jun. 5, 2003

(54) Nucleoside triphosphates and their incorporation into oligonucleotides

(76) Inventors: Leonid Beigelman, Longmont, CO (US); Shawn Zinnen, Denver, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/918,728

(22) Filed: Jul. 31, 2001

Related U.S. Application Data
(63) Continuation-in-part of application No. 09/825,805, filed on Apr. 4, 2001.
Application No. 09/825,805 is a continuation-in-part of application No. 09/578,223, filed on May 23, 2000.
Application No. 09/578,223 is a continuation-in-part of application No. 09/476,387, filed on Dec. 30, 1999.
Application No. 09/476,387 is a continuation-in-part of application No. 09/474,432, filed on Dec. 29, 1999.
Application No. 09/474,432 is a continuation-in-part of application No. 09/301,511, filed on Apr. 28, 1999.
Application No. 09/301,511 is a continuation-in-part of application No. 09/186,675, filed on Nov. 4, 1998, now Pat. No. 6,127,535.
(60) Provisional application No. 60/083,727, filed on Apr. 29, 1998.
(60) Provisional application No. 60/064,866, filed on Nov. 5, 1997.

(51) Int. Cl. C12Q 1/68; C07H 21/02

(52) U.S. Cl. 536/23.1; 435/6

(57) ABSTRACT

The present invention relates to novel nucleotide triphosphates, methods of synthesis and process of incorporating these nucleotide triphosphates into oligonucleotides, and isolation of novel nucleic acid catalysts (e.g., ribozymes or DNAzymes). Also, provided are the use of novel enzymatic nucleic acid molecules to inhibit gene expression and their applications in human therapy.

### RELATED APPLICATIONS

This patent application is a continuation-in-part of Beigelman et al., U.S. Ser. No. 09/825,805 filed Apr. 4, 2001, which is a continuation-in-part of Beigelman et al., U.S. Ser. No. 09/578,223 filed May 23, 2000, which is a continuation-in-part of Beigelman et al., U.S. Ser. No. 09/476,387 filed Dec. 30, 1999, which is a continuation-in-part of Beigelman et al., U.S. Ser. No. 09/474,432 filed Dec. 29, 1999,which is a continuation in part of Beigelman et al., U.S. Ser. No. 09/301,511 filed Apr. 28, 1999, which is a continuation-in-part of Beigelman et al., U.S. Ser. No. 09/186,675 filed Nov. 4, 1998 now U.S. Pat. No. 6,127,535, and claims the benefit of Beigelman et al., USS No. 60/083,727, filed Apr. 29, 1998, and Beigelman et al., USS No. 60/064,866 filed Nov. 5, 1997, all of these earlier applications are entitled “NUCLEOTIDE TRIPHOSPHATES AND THEIR INCORPORATION INTO OLIGONUCLEOTIDES”. Each of these applications is hereby incorporated by reference herein in its entirety, including the drawings.

### BACKGROUND OF THE INVENTION

This invention relates to novel nucleotide triphosphates (NTPs); methods for synthesizing nucleotide triphosphates; methods for incorporation of novel nucleotide triphosphates into oligonucleotides, and novel enzymatic nucleic acid molecules. The invention further relates to incorporation of nucleotide triphosphates into nucleic acid molecules using polymerases under several novel reaction conditions.

The following is a brief description of nucleotide triphosphates. This summary is not meant to be complete, but is provided only to assist understanding of the invention that follows. This summary is not an admission that all of the work described below is prior art to the claimed invention.

The synthesis of nucleotide triphosphates and their incorporation into nucleic acids using polymerase enzymes has greatly assisted in the advancement of nucleic acid research. The polymerase enzyme utilizes nucleotide triphosphates as precursor molecules to assemble oligonucleotides. Each nucleotide is attached by a phosphodiester bond formed through nucleophilic attack by the 3′ hydroxyl group of the oligonucleotide's last nucleotide onto the 5′ triphosphate of the next nucleotide. Nucleotides are incorporated one at a time into the oligonucleotide in a 5′ to 3′ direction. This process allows RNA to be produced and amplified from virtually any DNA or RNA templates.

Most natural polymerase enzymes incorporate standard nucleotide triphosphates into nucleic acid. For example, a DNA polymerase incorporates dATP, dTTP, dCTP, and dGTP into DNA and an RNA polymerase generally incorporates ATP, CTP, UTP, and GTP into RNA. There are however, certain polymerases that are capable of incorporating non-standard nucleotide triphosphates into nucleic acids (Joyce, 1997, PNAS 94, 1619-1622, Huang et al., Biochemistry 36, 8231-8242).

Before nucleosides can be incorporated into RNA transcripts using polymerase enzymes they must first be converted into nucleotide triphosphates which can be recognized by these enzymes. Phosphorylation of unblocked nucleosides by treatment with POCl₃ and trialkyl phosphates was shown to yield nucleoside 5′-phosphorodichloridates (Yoshikawa et al., 1969, Bull. Chem. Soc.(Japan) 42, 3505). Adenosine or 2′-deoxyadenosine 5′-triphosphate was synthesized by adding an additional step consisting of treatment with excess tri-n-butylammonium pyrophosphate in DMF followed by hydrolysis (Ludwig, 1981, Acta Biochim. et Biophys. Acad. Sci. Hung. 16, 131-133).

Non-standard nucleotide triphosphates are not readily incorporated into RNA transcripts by traditional RNA polymerases. Mutations have been introduced into RNA polymerase to facilitate incorporation of deoxyribonucleotides into RNA (Sousa & Padilla, 1995, EMBO J. 14,4609-4621, Bonner et al., 1992, EMBO J. 11, 3767-3775, Bonner et al., 1994, J. Biol. Chem. 42, 25120-25128, Aurup et al., 1992, Biochemistry 31, 9636-9641).

McGee et al., International PCT Publication No. WO 95/35102, describes the incorporation of 2′-NH₂-NTP's, 2′-F-NTP's, and 2′-deoxy-2′-benzyloxyamino UTP into RNA using bacteriophage T7 polymerase.

Wieczorek et al., 1994, Bioorganic & Medicinal Chemistry Letters 4, 987-994, describes the incorporation of 7-deaza-adenosine triphosphate into an RNA transcript using bacteriophage T7 RNA polymerase.

Lin et al., 1994, Nucleic Acids Research 22, 5229-5234, reports the incorporation of 2′-NH₂-CTP and 2′-NH₂-UTP into RNA using bacteriophage T7 RNA polymerase and polyethylene glycol containing buffer. The article describes the use of the polymerase synthesized RNA for in vitro selection of aptamers to human neutrophil elastase (HNE).

### SUMMARY OF THE INVENTION

The present invention features compositions and methods capable of modulating and/or diagnosing gene expression, useful in pharmaceutical, agriculture, research and diagnostic applications.

In one embodiment, the invention features a nucleic acid molecule with catalytic activity having Formula I:

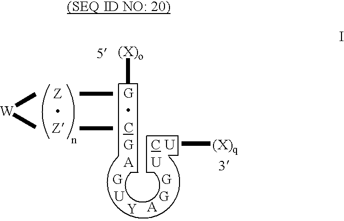

In the formula shown above X, Y, and Z represent independently a nucleotide or a non-nucleotide linker, which can be same or different; • indicates hydrogen bond formation between two adjacent nucleotides which can be present or absent; Z′ is a nucleotide complementary to Z; q is an integer greater than or equal to 3 and preferably less than 20, more specifically 4, 5, 6, 7, 8, 9, 10, 11, 12, or 15; n is an integer from 0 to about 10, more specifically 0, 1, 2, 3, 4, 5, 6, or 7; o is an integer greater than or equal to 3 and preferably less than 20, more specifically 3, 4, 5, 6, 7, 8, 9, 10, 11, 12, or 15; q and o can be the same length (q=o) or different lengths (q≠o); each X(q) and X(o) are oligonucleotides which are of sufficient length to stably interact independently with a target nucleic acid sequence (the target can be an RNA, DNA or RNA/DNA mixed polymers); X(o) preferably has a G at the 3′-end, X(q) preferably has a G at the 5′-end; W is a linker of ≧2 nucleotides in length or can be a non-nucleotide linker less than about 200 atoms in length; Y is a linker of ≧1 nucleotides in length, preferably G, 5′-CA-3′, or 5′-CAA-3′, or can be a non-nucleotide linker less than about 200 atoms in length; A, U, C, and G represent nucleotides; G is a nucleotide, preferably 2′-O-methyl, 2′-deozy-2′-fluoro, or 2′-OH; A is a nucleotide, preferably 2′-O-methyl, 2′-deozy-2′-fluoro, or 2′-OH; U is a nucleotide, preferably 2′-O-methyl, 2′-deozy-2′-fluoro, or 2′-OH; C represents a cytidine nucleotide, preferably 2′-amino (e.g., 2′-NH₂ or 2′-O—NH₂, and represents a chemical linkage (e.g. a phosphate ester linkage, amide linkage, phosphorothioate, phosphorodithioate or others known in the art).

In another embodiment, Z′ and Z of a compound having Formula I of the invention comprise G and C respectively, wherein G is a guanosine nucleotide and C is a cytidine nucleotide, either natuarally occuring or chemically modified at the sugar, phosphate, or nucleotide base.

In one embodiment, W of a compound having Formula I of the invention comprises 5′-GAAA-3′, wherein G is a guanosine nucleotide and A is an adenosine nucleotide, either natuarally occurring or chemically modified at the sugar, phosphate, or nucleotide base.

In another embodiment, Z′ and Z of a compound having Formula I of the invention are absent and W comprises 5′-UUAA-3′, 5′-CCGG-3′, 5′-AUAA-3′, 5′-GUAA-3′, 5′-GAUC-3′, 5′-UCGA-3′, 5′-UAUA-3′, wherein G is a guanosine nucleotide, C is a cytidine nucleotide, A is an adenosine nucleotide, and U is a uridine nucleotide, either natuarally occring or chemically modified at the sugar, phosphate, or nucleotide base.

In one embodiment, a compound having Formula I of the invention comprises between about one and about thirty-four, between about two and about twenty-one, or between about four and about eleven phosphorothioate intemucleotide linkages. In another embodiment, the phosphorothioate internucleotide linkages are at the 5′ end of the compound having Formula I of the invention.

In another embodiment, a compound having Formula I of the invention comprises a 3′-inverted abasic moiety.

In one embodiment, a compound having Formula I of the invention comprises between about twenty and about twenty-nine 2′-O-methyl nucleotides. In another embodiment, a compound having Formula I of the invention comprises between zero and about seven ribonucleotides.

In another embodiment, a compound having Formula I of the invention comprises an allozyme. In yet another embodiment, a compound having Formula I of the invention comprises a halfzyme.

In another embodiment, the invention features nucleic acid based techniques (e.g., enzymatic nucleic acid molecules), antisense nucleic acids, 2-5A antisense chimeras, triplex DNA, antisense nucleic acids containing RNA cleaving chemical groups) isolated using the methods described in this invention and methods for their use to diagnose, down regulate or inhibit gene expression.

In one embodiment, the invention features enzymatic nucleic acid molecules targeted against HER2 RNA, specifically including enzymatic nucleic acid molecules in the Zinzyme motif.

Targets, for example HER2, for useful enzymatic nucleic acids and antisense nucleic acids can be determined, for example, as described in Draper et al., WO 93/23569; Sullivan et al., WO 93/23057; Thompson et al., WO 94/02595; Draper et al., WO 95/04818; McSwiggen et al., U.S. Pat. Nos. 5,525,468 and 5,646,042, all are hereby incorporated by reference herein in their totalities. Other examples include the following PCT applications, which concern inactivation of expression of disease-related genes: WO 95/23225, and WO 95/13380; all of which are incorporated by reference herein.

In one embodiment, the invention features a method of inhibiting expression of HER2 in a cell, comprising the step of contacting the cell with a chemotherapeutic agent and an enzymatic nucleic acid molecule having a Formula I under conditions suitable for the inhibition of expression of HER2.

In another embodiment, the invention features a method of treatment of a patient having a condition associated with the level of HER2, wherein the patient is administered a chemotherapeutic agent and an enzymatic nucleic acid molecule having a Formula I under conditions suitable for the treatment.

In another embodiment, the invention features a method for treating conditions associated with the level of HER2 gene using a chemotherapeutic agent in combination with an enzymatic nucleic acid molecule having a Formula I under conditions suitable for the treatment.

In a preferred embodiment, the invention features a method for treating cancer using a chemotherapeutic agent in combination with an enzymatic nucleic acid molecule having a Formula I under conditions suitable for the treatment.

Suitable chemotherapeutic agents include chemotherapeutic agents selected from the group consisting of Paclitaxel, Doxorubicin, Cisplatin, and Herceptin.

In another embodiment, enzymatic nucleic acid molecules of the instant invention are used to treat cancers selected from the group consisting of breast cancer, non-small cell lung cancer, bladder cancer, prostate cancer, and pancreatic cancer.

The enzymatic nucleic acid molecules of Formula I can independently comprise a cap structure which may independently be present or absent.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover.

By “stably interact” is meant interaction of the oligonucleotides with target nucleic acid (e.g., by forming hydrogen bonds with complementary nucleotides in the target under physiological conditions).

By “chimeric nucleic acid molecule” or “chimeric oligonucleotide” is meant that the molecule can be comprised of both modified or unmodified DNA or RNA.

By “cap structure” is meant chemical modifications, which have been incorporated at a terminus of the oligonucleotide. These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap is selected from the group consisting of inverted abasic residue (moiety), 4′,5′-methylene nucleotide, 1-(beta-D-erythrofuranosyl) nucleotide, 4′-thio nucleotide, carbocyclic nucleotide; 1,5-anhydrohexitol nucleotide; L-nucleotides; alpha-nucleotides, modified base nucleotide, phosphorodithioate linkage, threo-pentofuranosyl nucleotide, acyclic 3′,4′-seco nucleotide, acyclic 3,4-dihydroxybutyl nucleotide, acyclic 3,5-dihydroxypentyl nucleotide, 3′-3′-inverted nucleotide moiety, 3′-3′-inverted abasic moiety; 3′-2′-inverted nucleotide moiety; 3′-2′-inverted abasic moiety; 1,4-butanediol phosphate, 3′-phosphoramidate, hexylphosphate, aminohexyl phosphate; 3′-phosphate, 3′-phosphorothioate, phosphorodithioate, or bridging or non-bridging methylphosphonate moiety (for more details, see Beigelman et al., International PCT publication No. WO 97/26270, incorporated by reference herein).

In another embodiment, the 3′-cap can be selected from a group consisting of 4′,5′-methylene nucleotide; 1-(beta-D-erythrofuranosyl) nucleotide; 4′-thio nucleotide; carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide; 5′-5′-inverted nucleotide moiety; 5′-5′-inverted abasic moiety; 5′-phosphoramidate; 5′-phosphorothioate; 1,4-butanediol phosphate 5′-amino; bridging and/or non-bridging 5′-phosphoramidate, phosphorothioate and/or phosphorodithioate; bridging or non-bridging methylphosphonate and 5′-mercapto moieties (for more details, see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine. The terms “abasic” or “abasic nucleotide” as used herein encompass sugar moieties lacking a base or having other chemical groups in place of base at the 1′ position.

In connection with 2′-modified nucleotides as described for the present invention, by “amino” is meant 2′-NH₂ or 2′-O—N₁H₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

By “modulate” is meant that the expression of the gene, or level of RNAs or equivalent RNAs encoding one or more protein subunits, or activity of one or more protein subunit(s) is up-regulated or down-regulated, such that the expression, level, or activity is greater than or less than that observed in the absence of the nucleic acid molecules of the invention.

By “inhibit”, “down-regulate”, or “reduce”, it is meant that the expression of the gene, or level of RNAs or equivalent RNAs encoding one or more protein subunits, or activity of one or more protein subunits, such as BACE, ps-2, or APP, is reduced below that observed in the absence of the nucleic acid molecules of the invention. In one embodiment, inhibition. down-regulation or reduction with enzymatic nucleic acid molecule preferably is below that level observed in the presence of an enzymatically inactive or attenuated molecule that is able to bind to the same site on the target RNA, but is unable to cleave that RNA. In another embodiment, inhibition, down-regulation, or reduction with antisense oligonucleotides is preferably below that level observed in the presence of, for example, an oligonucleotide with scrambled sequence or with mismatches. In another embodiment, inhibition, down-regulation, or reduction of BACE, ps-2, or APP with the nucleic acid molecule of the instant invention is greater in the presence of the nucleic acid molecule than in its absence.

By “up-regulate” is meant that the expression of the gene, or level of RNAs or equivalent RNAs encoding one or more protein subunits, or activity of one or more protein subunits, such as BACE, PS-2, or APP subunit(s), is greater than that observed in the absence of the nucleic acid molecules of the invention. For example, the expression of a gene, such as BACE, PS-2, or APP gene, can be increased in order to treat, prevent, ameliorate, or modulate a pathological condition caused or exacerbated by an absence or low level of gene expression.

The term “nucleotide” as used herein is as recognized in the art to include natural bases (standard), and modified bases well known in the art. Such bases are generally located at the 1′ position of a sugar moiety. Nucleotides generally include a base, a sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, Ann. Rev. Med. Chem. 30:285-294; Eckstein et al., International PCT Publication No. WO 92/07065; Usman et al., International PCT Publication No. WO 93/15187; all of which are hereby incorporated by reference herein). There are several examples of modified nucleic acid bases known in the art, e.g., as recently summarized by Limbach et al., 1994, Nucleic Acids Res. 22, 2183. Some of the non-limiting examples of base modifications that can be introduced into nucleic acids without significantly effecting their catalytic activity include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine) and others (Burgin et al., 1996, Biochemistry, 35, 14090).

By “modified bases” in this aspect is meant nucleotide bases other than adenine, guanine, cytosine, thymine, and uracil at 1′ position or their equivalents; such bases may be used within the catalytic core of an enzymatic nucleic acid molecule and/or in the substrate-binding regions of such a molecule. Such modified nucleotides include dideoxynucleotides which have pharmaceutical utility well known in the art, as well as utility in basic molecular biology methods such as sequencing.

By “ribonucleotide” is meant a nucleotide with a hydroxyl group at the 2′ position of a D-ribo-furanose moiety.

By “unmodified nucleoside” or “unmodified nucleotide” is meant one of the bases adenine, cytosine, guanine, uracil joined to the 1′ carbon of β-D-ribo-furanose with substitutions on either moiety.

By “modified nucleoside” or “modified nucleotide” is meant any nucleotide base which contains a modification in the chemical structure of an unmodified nucleotide base, sugar and/or phosphate.

By “pyrimidines” is meant nucleotides comprising modified or unmodified derivatives of a six membered pyrimidine ring. An example of a pyrimidine is modified or unmodified uridine.

By “nucleotide triphosphate” or “NTP” is meant a nucleoside bound to three inorganic phosphate groups at the 5′ hydroxyl group of the modified or unmodified ribose or deoxyribose sugar where the 1′ position of the sugar may comprise a nucleic acid base or hydrogen. The triphosphate portion may be modified to include chemical moieties which do not destroy the functionality of the group (i.e., allow incorporation into an RNA molecule).

By “enhancer of modified NTP incorporation” is meant a reagent which facilitates the incorporation of modified nucleotides into a nucleic acid transcript by an RNA polymerase. Such reagents include, but are not limited to, methanol, LiCl, polyethylene glycol (PEG), diethyl ether, propanol, methyl amine, ethanol, and the like.

By “antisense” it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004; Agrawal et al., U.S. Pat. No. 5,591,721; Agrawal, U.S. Pat. No. 5,652,356). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

The term “2-5A chimera” as used herein refers to an oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113). By “2-5A antisense chimera” it is meant, an antisense oligonucleotide containing a 5′ phosphorylated 2′-5′-linked adenylate residues. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300).

The term “allozyme” as used herein refers to an allosteric enzymatic nucleic acid molecule, see for example see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

By “halfzyme” is meant an enzymatic nucleic acid molecule assembled from two or more nucleic acid components. The enzymatic nucleic acid in the halfzyme configuration is active when all the necessary components interact with each other. The halfzyme construct can be engineered to have a component lacking from the structure or sequence of the enzymatic nucleic acid molecule such that enzymatic activity is inhibited. In the presence of the target signaling agent, the required component for enzymatic activity is provided such that the halfzyme is catalytically active.

By “triplex forming oligonucleotides (TFO)” it is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504).

By “oligonucleotide” as used herein is meant a molecule having two or more nucleotides. The polynucleotide can be single-, double- or multiple-stranded and can have modified or unmodified nucleotides or non-nucleotides or various mixtures and combinations thereof.

By “nucleic acid catalyst” is meant a nucleic acid molecule capable of catalyzing (altering the velocity and/or rate of) a variety of reactions including the ability to repeatedly cleave other separate nucleic acid molecules (endonuclease activity) in a nucleotide base sequence-specific manner. Such a molecule with endonuclease activity can have complementarity in a substrate binding region to a specified gene target, and also has an enzymatic activity that specifically cleaves RNA or DNA in that target. That is, the nucleic acid molecule with endonuclease activity is able to intramolecularly or intermolecularly cleave RNA or DNA and thereby inactivate a target RNA or DNA molecule. This complementarity functions to allow sufficient hybridization of the enzymatic RNA molecule to the target RNA or DNA to allow the cleavage to occur. 100% complementarity is preferred, but complementarity as low as 50-75% can also be useful in this invention. The nucleic acids can be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme, finderon or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, 260 JAMA 3030).

By “enzymatic portion” or “catalytic domain” is meant that portion/region of the enzymatic nucleic acid molecule essential for cleavage of a nucleic acid substrate.

By “substrate binding arm” or “substrate binding domain” is meant that portion/region of an enzymatic nucleic acid molecule which is complementary to (i.e., able to base-pair with) a portion of its substrate. Generally, such complementarity is 100%, but can be less. For example, as few as 10 bases out of 14 can be base-paired. That is, these arms contain sequences within a enzymatic nucleic acid molecule which are intended to bring enzymatic nucleic acid molecule and target together through complementary base-pairing interactions. The enzymatic nucleic acid molecule of the invention can have binding arms that are contiguous or non-contiguous and can be varying lengths. The length of the binding arm(s) are preferably greater than or equal to four nucleotides; specifically 12-100 nucleotides; more specifically 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like). Binding arms can be complementary to the specified substrate, to a portion of the indicated substrate, to the indicated substrate sequence and additional adjacent sequence, or a portion of the indicated sequence and additional adjacent sequence.

By “nucleic acid molecule” as used herein is meant a molecule having nucleotides. The nucleic acid molecule can be single-, double- or multiple-stranded and can comprise modified or unmodified nucleotides or non-nucleotides or various mixtures and combinations thereof. In preferred embodiments of the present invention, a nucleic acid molecule, e.g., an antisense molecule, a triplex DNA, or an enzymatic nucleic acid molecule, is greater than about 12 nucleotides in length. In particularly preferred embodiments, the nucleic acid molecule is between 12 and 100 nucleotides in length, e.g., in specific embodiments 35, 36, 37, or 38 nucleotides in length for particular ribozymes. In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit in particularly preferred embodiments, the upper limit of the length range in some preferred embodiments can be, for example, 30, 40, 50, 60, 70, or 80 nucleotides. Thus, for any of the length ranges, the length range for particular embodiments has a lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the specific lengths within the range specified above, for example, 21 nucleotides in length.

By “complementarity” is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well-known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785. A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

As used herein “cell” is used in its usual biological sense, and does not refer to an entire multicellular organism. The cell can, for example, be in vitro, e.g., in cell culture, or present in a multicellular organism, including, e.g., birds, plants and mammals, such as humans, cows, sheep, apes, monkeys, swine, dogs, cats, rats and mice. In another aspect, the invention provides mammalian cells containing one or more nucleic acid molecules and/or expression vectors of this invention. The one or more nucleic acid molecules can independently be targeted to the same or different sites.

### DESCRIPTION OF THE PREFERRED EMBODIMENTS

The drawings will first briefly be described.

### DRAWINGS

FIG. 1 shows a schematic of the enzymatic nucleic acid selection protocol. (A) The pool RNA is loaded onto column #1. After binding to the target RNA and incubating in the presence of magnesium and calcium, active motifs are able to cleave at the indicated site. These molecules are eluted and then passed over column #2. Molecules not bound to product are washed off this second column, and the desired molecules are then eluted with urea at 90° C. The eluted desired molecules are subsequently reverse transcribed, PCR amplified and transcribed into an enriched pool for the next cycle of selection. (B) The modified NTP's utilized in the transcriptions (top to bottom): 2′-F-5-[(N-imidazole-4-acetyl) propylamine]-UTP, 2′-F-UTP, 2′—NH₂-CTP and 2′-F-ATP.

FIGS. 2a-b shows predicted structures of the primary selection isolates. The arrow on the substrate strand indicates the cleavage site. The six motifs shown can all cleave with minimization in the binding arms (7/7) without significantly affecting rate. All cytidine residues are 2′-amino modified. The arrows in the diagrams shown above indicate the cleavage site within the substrate. Enzymatic NA refers to the enzymatic nucleic acid molecule.

FIGS. 3a-d shows the dependence of Zinzyme activity on either magnesium or calcium concentrations. The arrow indicates the activity as assessed under the selection conditions were both magnesium and calcium were present at 1 mM.

FIG. 4 shows predicted structures of motifs A5, B6 and their respective truncated variants. Design of the enzymatically transcribed sequences shown was the first step in testing the activity of these motifs with shortened binding arms and stem II sequences.

FIG. 5 shows the stability of Zinzymes in Human Serum. Four Zinzymes were 5 end labeled and incubated in fresh human serum. a,c,g,u=2′-OMe, G=2′-OH—G, C=2′-NH₂-C, u=2′-F—U, B=inverted abasic and gugc=phosphorothioate linkages. Decay rates were obtained by fitting to an exponential equation and were converted to half-life; t_{½}=ln2/rate.

FIGS. 6a-c shows the kinetic evaluation of Zinzyme. (A) Single turnover kinetic measurements. The inset shows the cleavage profiles with increasing concentrations of ribozyme. The points were fit to a first order exponential equation to extract the observed rate constants. The rates were replotted as a function of ribozyme concentration to obtain the values: k_{cat}=0.07 min-1 and Kₘ=70 nM. (B) Multiple turnover kinetic measurements. 50 nM=Δ, and 100 nM=O ribozyme was reacted in an excess of substrate. Lines were fit by least squared regression analysis. Slope/[ribozyme]=multiturnover rate=0.013 min⁻¹. (C) Predicted structure of Zinzyme in the generalized forrnat. In the kinetic evaluation the substrate is the same 15-mer fragment of the Kras mRNA used for previous testing.

FIG. 7 is a diagram of a novel 35 nucleotide enzymatic nucleic acid motif (Zinzyme) which was identified using in vitro methods described in the instant invention. The molecule shown is only exemplary. The 5′ and 3′ terminal nucleotides (referring to the nucleotides of the substrate binding arms rather than merely the single terminal nucleotide on the 5′ and 3′ ends) can be varied so long as those portions can base-pair with target substrate sequence. In addition, the guanosine (G) shown at the cleavage site of the substrate can be changed to other nucleotides so long as the change does not eliminate the ability of enzymatic nucleic acid molecules to cleave the target sequence. Substitutions in the nucleic acid molecule and/or in the substrate sequence can be readily tested, for example, as described herein.

FIGS. 8a-b is a diagram of “no-ribo” Zinzymes.

FIG. 9 is a graph showing cleavage reactions with Zinzymes under differing divalent metal concentrations.

FIG. 10 is a diagram of differing Zinzymess with varying ribo content and their relative rates of catalysis.

FIG. 11 shows a non-limiting example of the replacement of a 2′-O-methyl 5′-CA-3′ with a ribo G in the class II (zinzyme) motif. The representative motif shown for the purpose of the figure is a “seven-ribo” zinzyme motif, however, the interchangeability of a G and a CA in the position shown in FIG. 11 of the Zinzyme motif extends to any combination of 2-O-methyl and ribo residues. For instance, a 2′-O-methyl G can replace the 2′-O-methyl 5′-CA-3′ and vise versa.

FIG. 12a-b shows the design of a combinatorial study to determine optimal Zinzyme tetraloop sequences by systematically replacing the stem II region of a Zinzyme with the tetraloop (FIG. 12a). All possible four nucleotide combinations for the tetraloop were tested (FIG. 12b).

FIG. 13 shows sequences and cleavage data of Zinzymes with varing numbers of phosphorothioate internucleotide linkages.

FIG. 14 shows sequences and cleavage data of Zinzymes with phosphorothioate, 2′-O-methyl or 2′-O-allyl nucleotide modification.

FIGS. 15a-b shows the structure of a Zinzyme 5′-folic acid conjugate (FIG. 15a) and cleavage data of the Zinzyme 5′-folic acid conjugate (FIG. 15b).

Transcription Conditions

Incorporation rates of modified nucleotide triphosphates into oligonucleotides can be increased by adding to traditional buffer conditions, several different enhancers of modified NTP incorporation. Applicant has utilized methanol and LiCl in an attempt to increase incorporation rates of dNTP using RNA polymerase. These enhancers of modified NTP incorporation can be used in different combinations and ratios to optimize transcription. Optimal reaction conditions differ between nucleotide triphosphates and can readily be determined by standard experimentation. Overall, however, Applicant has found that inclusion of enhancers of modified NTP incorporation such as methanol or inorganic compound such as lithium chloride increase the mean transcription rates.

Mechanism of action of Nucleic Acid Molecules of the Invention

Enzymatic Nucleic Acid:

In general, enzymatic nucleic acids act by first binding to a target RNA. Such binding occurs through the target-binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA destroys its ability to direct synthesis of an encoded protein. After an enzymatic nucleic acid has bound and cleaved its RNA target, it is released from that RNA to search for another target and can repeatedly bind and cleave new targets.

The enzymatic nature of an enzymatic nucleic acid has significant advantages, such as the concentration of enzymatic nucleic acid molecules necessary to affect a therapeutic treatment is lower. This advantage reflects the ability of the enzymatic nucleic acid molecules to act enzymatically. Thus, a single enzymatic nucleic acid molecule can cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of enzymatic nucleic acid molecules.

Nucleic acid molecules having an endonuclease enzymatic activity are able to repeatedly cleave other separate RNA molecules in a nucleotide base sequence-specific manner. With proper design, such enzymatic nucleic acid molecules can be targeted to RNA transcripts, and efficient cleavage achieved in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 infra).

Because of their sequence-specificity, trans-cleaving enzymatic nucleic acid molecules show promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited.

Enzymatic nucleic acid molecules of the invention that are allosterically regulated (“allozymes”) can be used to down-regulate gene expression, for example HER2 expression. These allosteric enzymatic nucleic acids or allozymes (see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, for example, mutant HER2 protein, wild-type HER2 protein, mutant HER2 RNA, wild-type HER2 RNA, other proteins and/or RNAs involved in HER2 activity, compounds, metals, polymers, molecules and/or drugs that are targeted to HER2 expressing cells etc., which in turn modulates the activity of the enzymatic nucleic acid molecule. In response to interaction with a predetermined signaling agent, the allosteric enzymatic nucleic acid molecule's activity is activated or inhibited such that the expression of a particular target is selectively down-regulated. The target can comprise wild-type HER2, mutant HER2, a component of HER2, and/or a predetermined cellular component that modulates HER2 activity. In a specific example, allosteric enzymatic nucleic acid molecules that are activated by interaction with a RNA encoding HER2 protein are used as therapeutic agents in vivo. The presence of RNA encoding the HER2 protein activates the allosteric enzymatic nucleic acid molecule that subsequently cleaves the RNA encoding HER2 protein resulting in the inhibition of HER2 protein expression. In this manner, cells that express the the HER2 protein are selectively targeted.

In another non-limiting example, an allozyme can be activated by a HER2 protein, peptide, or mutant polypeptide that caused the allozyme to inhibit the expression of HER2 gene, by, for example, cleaving RNA encoded by HER2 gene. In this non-limiting example, the allozyme acts as a decoy to inhibit the function of HER2 and also inhibit the expression of HER2 once activated by the HER2 protein.

Synthesis of Nucleic acid Molecules

Synthesis of nucleic acids greater than about 100 nucleotides in length is difficult using automated methods, and the therapeutic cost of such molecules is prohibitive. In this invention, small nucleic acid motifs (“small” refers to nucleic acid motifs less than about 100 nucleotides in length, preferably less than about 80 nucleotides in length, and more preferably less than about 50 nucleotides in length; e.g., antisense oligonucleotides, hammerhead or the hairpin ribozymes) are preferably used for exogenous delivery. The simple structure of these molecules increases the ability of the nucleic acid to invade targeted regions of RNA structure. Exemplary molecules of the instant invention were chemically synthesized, and others can similarly be synthesized. Oligodeoxyribonucleotides were synthesized using standard protocols as described in Caruthers et al., 1992, Methods in Enzymology 211,3-19, which is incorporated herein by reference.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses were conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table 11 outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M =6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, were 97.5-99%. Other oligonucleotide synthesis reagents for the 394 Applied Biosystems, Inc. synthesizer; detritylation solution was 3% TCA in methylene chloride (ABI); capping was performed with 16% N-methyl imidazole in THF (ABI) and 10% acetic anhydride/10% 2,6-lutidine in THF (ABI); oxidation solution was 16.9 mM I₂, 49 mM pyridine, 9% water in THF (PERSEPTIVE™). Burdick & Jackson Synthesis Grade acetonitrile was used directly from the reagent bottle. S-Ethyltetrazole solution (0.25 M in acetonitrile) was made up from the solid obtained from American International Chemical, Inc.

Deprotection of the RNA was performed using either a two-pot or one-pot protocol. For the two-pot protocol, the polymer-bound trityl-on oligoribonucleotide was transferred to a 4 mL glass screw top vial and suspended in a solution of 40% aq. methylamine (1 mL) at 65° C. for 10 min. After cooling to −20° C., the supernatant was removed from the polymer support. The support was washed three times with 1.0 mL of EtOH:MeCN:H20/3:1:1, vortexed and the supernatant was then added to the first supernatant. The combined supernatants, containing the oligoribonucleotide, were dried to a white powder. The base deprotected oligoribonucleotide was resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer was quenched with 1.5 M NH₄HCO₃.

Alternatively, for the one-pot protocol, the polymer-bound trityl-on oligoribonucleotide was transferred to a 4 mL glass screw top vial and suspended in a solution of 33% ethanolic methylamine/DMSO: 1/1 (0.8 mL) at 65° C. for 15 min. The vial was brought to r.t. TEA.3HF (0.1 mL) was added and the vial was heated at 65° C. for 15 min. The sample was cooled at −20° C. and then quenched with 1.5 M NH₄HCO₃.

For purification of the trityl-on oligomers, the quenched NH₄HCO₃ solution was loaded onto a C-18 containing cartridge that had been prewashed with acetonitrile followed by 50 mM TEAA. After washing the loaded cartridge with water, the RNA was detritylated with 0.5% TFA for 13 min. The cartridge was then washed again with water, salt exchanged with 1 M NaCl and washed with water again. The oligonucleotide was then eluted with 30% acetonitrile.

Inactive hammerhead ribozymes or binding attenuated control (BAC) oligonucleotides) were synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields were >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; see Wincott et al., supra, the totality of which is hereby incorporated herein by reference) and are re-suspended in water.

Optimizing Nucleic Acid Catalyst Activity

Catalytic activity of the enzymatic nucleic acid molecules described and identified using the methods of the instant invention, can be optimized as described by Draper et al., supra and using the methods well known in the art. The details will not be repeated here, but include altering the length of the enzymatic nucleic acid molecules' binding arms, or chemically synthesizing enzymatic nucleic acid molecules with modifications (base, sugar and/or phosphate) that prevent their degradation by serum ribonucleases and/or enhance their enzymatic activity (see e.g., Eckstein et al., International Publication No. WO 92/07065; Perrault et al., 1990 Nature 344, 565; Pieken et al., 1991 Science 253, 314; Usman and Cedergren, 1992 Trends in Biochem. Sci. 17, 334; Usman et al., International Publication No. WO 93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of enzymatic nucleic acid molecules). All these publications are hereby incorporated by reference herein. Modifications which enhance their efficacy in cells, as well as removal of bases from stem loop structures to shorten synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al, U.S. Pat. No. 5,627,053; Woolf et al., International PCT Publication No. WO 98/13526; Thompson et al., USS No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verna and Eckstein, 1998, Annu. Rev. Biochem., 67, 99-134; and Burlina et al., 1997, Bioorg. Med. Chem., 5, 1999-2010; all of these references are hereby incorporated by reference herein in their totalities). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorodithioate, and/or 5′-methylphosphonate linkages improves stability, too many of these modifications may cause some toxicity. Therefore, when designing nucleic acid molecules, the amount of these intemucleotide linkages should be minimized, but can be balanced to provide acceptable stability while reducing potential toxicity. The reduction in the concentration of these linkages should lower toxicity resulting in increased efficacy and higher specificity of these molecules.

Nucleic acid catalysts having chemical modifications which maintain or enhance enzymatic activity are provided. Such nucleic acid molecules are generally more resistant to nucleases than unmodified nucleic acid. Thus, in a cell and/or in vivo the activity may not be significantly lowered. As exemplified herein, such enzymatic nucleic acid molecules are useful in a cell and/or in vivo even if activity over all is reduced 10-fold (Burgin et al., 1996, Biochemistry, 35, 14090). Such enzymatic nucleic acid molecules herein are said to “maintain” the enzymatic activity.

Therapeutic nucleic acid molecules (e.g., enzymatic nucleic acid molecules and antisense nucleic acid molecules) delivered exogenously must optimally be stable within cells until translation of the target RNA has been inhibited long enough to reduce the levels of the undesirable protein. This period of time varies between hours to days depending upon the disease state. Clearly, these nucleic acid molecules must be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

By “enhanced enzymatic activity” is meant to include activity measured in cells and/or in vivo where the activity is a reflection of both catalytic activity and enzymatic nucleic acid molecules stability. In this invention, the product of these properties is increased or not significantly (less than 10-fold) decreased in vivo compared to unmodified enzymatic nucleic acid molecules.

In one embodiment, nucleic acid catalysts having chemical modifications which maintain or enhance enzymatic activity are provided. Such nucleic acid is also generally more resistant to nucleases than unmodified nucleic acid. Thus, in a cell and/or in vivo the activity may not be significantly lowered. As exemplified herein such enzymatic nucleic acid molecules are useful in a cell and/or in vivo even if activity over all is reduced 10-fold (Burgin et al., 1996, Biochemistry, 35, 14090). Such enzymatic nucleic acid molecules herein are said to “maintain” the enzymatic activity on all RNA enzymatic nucleic acid molecule.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecules motifs) and/or other chemical or biological molecules. The treatment of patients with nucleic acid molecules can also include combinations of different types of nucleic acid molecules. Therapies can be devised which include a mixture of enzymatic nucleic acid molecules (including different enzymatic nucleic acid molecules motifs), antisense and/or 2-5A chimera molecules to one or more targets to alleviate symptoms of a disease.

Administration of Nucleotide Mono, di or Triphosphates and Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules can be directly delivered ex vivo to cells or tissues with or without the aforementioned vehicles. Alternatively, the nucleic acid/vehicle combination can be locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which are incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged nucleotide mono, di or triphosphates of the invention can be administered and introduced into a patient by any standard means, such as those described above and other methods known in the art, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., ammonium, sodium, calcium, magnesium, lithium, tributylammoniun, and potassium salts.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered). For example, pharmacological compositions injected into the blood stream should be soluble. Other factors for pharmaceutical formulation are known in the art, and include, for example, considerations such as toxicity and formulations which impede or prevent the enzymatic nucleic acid molecule from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., NTP's, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which facilitates the association of drug with the surface of cells such as lymphocytes and macrophages is also useful. This approach can provide enhanced delivery of the drug to target cells by taking advantage of the specificity of macrophage and lymphocyte immune recognition of abnormal cells, such as cancer cells.

The invention also features compositions comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokineties and pharmacodynamics of drugs, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of these are incorporated by reference herein). Long-circulating liposomes are also likely to protect drugs from nuclease degradation to a greater extent compared to cationic liposomes, based on their ability to avoid accumulation in metabolically aggressive MPS tissues, such as the liver and spleen. All of these references are incorporated by reference herein.

The present invention also features compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. Suitable carriers can include, for example, preservatives, stabilizers, dyes and flavoring agents, such as sodium benzoate, sorbic acid and esters of p-hydroxybenzoic acid. Id. at 1449. In addition, antioxidants and suspending agents can be included in acceptable carriers.

By “patient” is meant an organism which is a donor or recipient of explanted cells or the cells themselves. “Patient” also refers to an organism or the cells of an organism to which the compounds of the invention can be administered. Preferably, the patient is a mammal, e.g., a human, primate or a rodent.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer. In a one aspect, the invention provides enzymatic nucleic acid molecules that can be delivered exogenously to specific cells as required.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapies and therapeutic compounds, The use of monoclonal antibodies, chemotherapy, radiation therapy, and analgesics are examples of treatments that can be used to increase the overall therapeutic effect of compound of the invention. The use of multiple compounds to treat an indication can increase the beneficial effects while reducing the presence of side effects. Examples of chemotherapeutic agents that can be combined with the nucleic acid molecules of the invention include, but are not limited to, Paclitaxel, Doxorubicin, Cisplatin, and/or antibodies such as Herceptin.

### EXAMPLES

The following are non-limiting examples showing the synthesis, incorporation and analysis of nucleotide triphosphates and activity of enzymatic nucleic acids of the instant invention.

Applicant synthesized pyrimidine nucleotide triphosphates using DMAP in the reaction. For purines, applicant utilized standard protocols previously described in the art (Yoshikawa et al supra;. Ludwig, supra). Described below is one example of a pyrimdine nucleotide triphosphate and one purine nucleotide triphosphate synthesis.

### Example 1

### Selection of the Zinzyme Enzymatic Nucleic Acid

An in vitro selection was designed to identify RNA-cleaving enzymatic nucleic acid molecules predisposed for function as a drug. The selection scheme required the catalyst to be trans-acting with phosphodiesterase activity targeting a fragment of the K-ras mRNA under simulated physiological conditions. To increase stabilization against nucleases and to offer the potential for improved functionality, modified sequence space was sampled by transcribing with the following NTP'S: 2′-F-ATP, 2′-F-UTP or 2′-F-5-[(N-imidazole-4-acetyl) propylamine]-UTP, 2′-NH₂-CTP, and GTP. Active motifs were identified and assessed for their modified NMP and divalent metal dependence. The minimization of the enzymatic nucleic acid's size and the ability to substitute 2′-OMe for 2′-F and 2′-NH₂ moieties yielded the motif from these selections most suited for both nuclease stability and therapeutic development. This motif utilizes only two 2′-NH₂-C's and functions as a 36-mer. The substrate sequence requirements were determined to be 5′-Y—G—H—3′. The half-life of the enzymatic nucleic acid in human serum is >100 hours. In physiologically relevant magnesium concentrations [˜1 mM] the enzymatic nucleic acid molecule has a k_{cat}=0.07 min⁻¹, and Kₘ=70 nM.

Selection

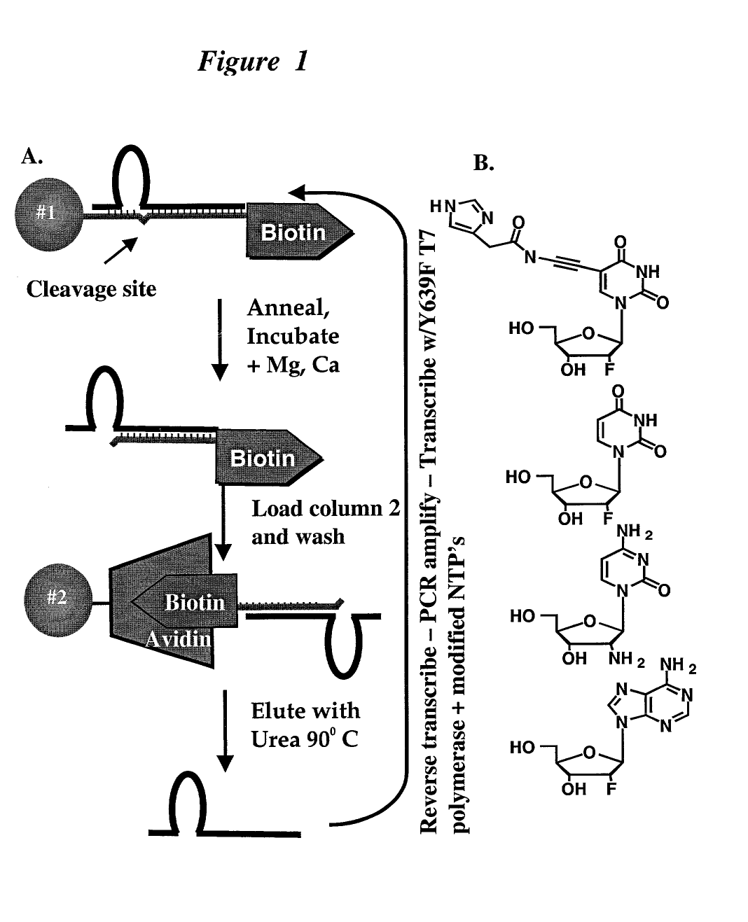

A schematic of the selection protocol is shown in FIG. 1. Initial pools containing >7×10¹³ molecules of modified RNA were transcribed with the following NTPs: 2′-F-ATP, 2′-F-UTP, 2′-NH₂-CTP, and GTP (selection A), or 2′-F-ATP, 2′-F-5-[(N-imidazole-4-acetyl) propylamine]-UTP, 2′-NH₂-CTP, and GTP (selection B). The modifications were included to provide stabilization against nucleases and to offer alternative chemical functionalities. GTP was maintained as a ribonucleotide for ease of radioisotope labeling and tracking of the selection's progression. The pool RNAs contained a 35 nucleotide random region surrounded by two binding arms recognizing a fragment of the Kras MRNA. The weak binding arm (8 base pairs K_{d}=3.5 nM; dissociation T_{½}=2minutes at 37° C., facilitated post-cleavage dissociation from the column bound substrate while the tight binding arm (21 base pairs K_{d}=1.6×10⁻²¹ nM; Dissociation T_{½}=87×10¹⁵ years at 37° C.) maintained association to the biotinylated product. The first column was generated by coupling to the resin (via a thiol linkage to the 3′-terminus) a fragment of the K-ras gene that possessed the 5′-biotin moiety. The K-ras sequences were complementary to the weak and tight binding arms of the RNA pool and maintained a bulged G nucleotide between those binding arms. The RNA was loaded on the first column in 20 mM HEPES pH 7.4, 10 mM NaCl, 14 mM KCl (termed PB, for simulated Physiological Buffer) at 37° C. (selection A and B) or 25° C. (selection C). After sufficient washing the cleavage reaction was triggered by the addition of a mixture of 1 mM MgCl₂ and 1 mM CaCl₂ in PB (subsequently termed PBM for PB plus divalent Metal ions) and incubated for 20 minutes. RNAs eluted in the presence of divalent metal ions were then passed over a neutravidin resin to capture active molecules through their bound biotinylated products. The active molecules were eluted from the neutravidin resin with 90° C. urea, RT-PCR amplified and subjected to repeated rounds of selection, as shown in FIG. 1.

In the selections labeled A and B, all steps were performed at 37° C. to mimic physiological conditions. To assess the effect of temperature, a parallel selection was performed at 25° C. (selection C) with all other parameters identical to the B selection. Activity was detected in all pools after eight cycles, and increased over the next two cycles. Cloning, sequencing and activity assays of isolates provided six motifs that were used for synthetic mutagenesis at a level of 36% per position. Two motifs from selection A, three from selection B and one from selection C were considered parental motifs; see Table 1. Mutagenized pools were then subjected to more stringent selective pressure in an attempt to find a more active catalyst. The time of exposure to divalent metals was reduced from 20 minutes to 10 seconds. When no signal was detected for eight rounds, the reaction time was then increased from 10 seconds to 10 minutes and activity was immediately detected. This result suggested that while active molecules were always present, none possessed activity significantly above that of the parental rates. Cloning, sequencing and activity assays of individual isolates were performed. The single turnover cleavage rate measurements of the pools and of individual isolates show a range of activity, k=0.01 to 0.1 min⁻¹ in PBM at 37° C. Due to the equivalent cleavage rates for the parental and reselected pools the reselected isolates were not assessed kinetically. Thus, reselection primarily generated variant motifs of comparable activities and provided pseudo-phylogenetic information to aid in structural predictions. Many possible new motifs of comparable activity were identified.

Sequences and Structural Predictions

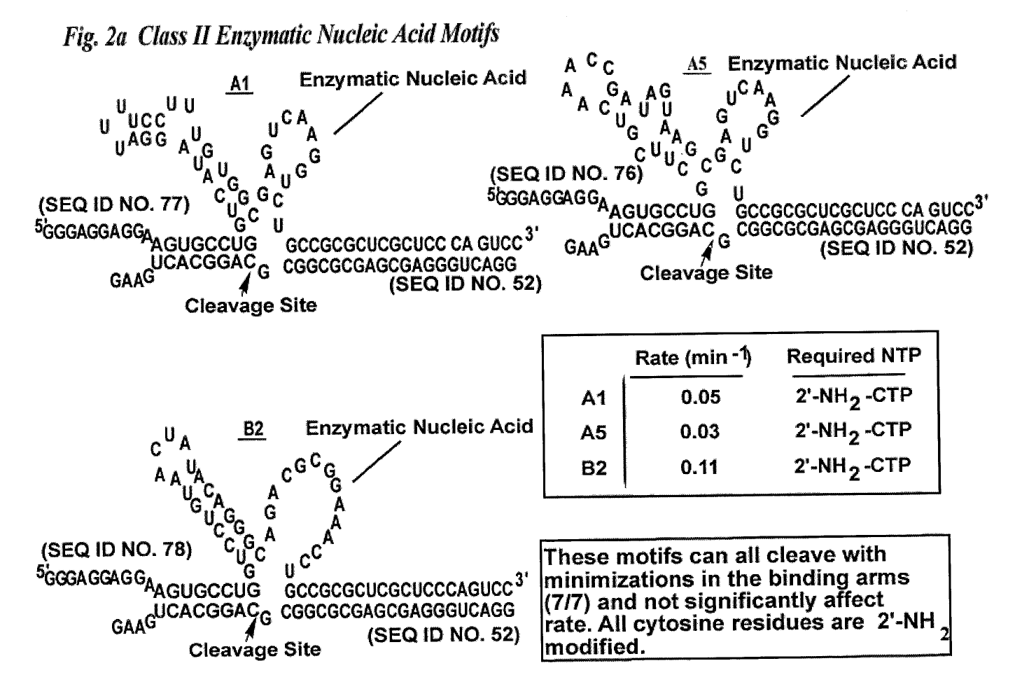

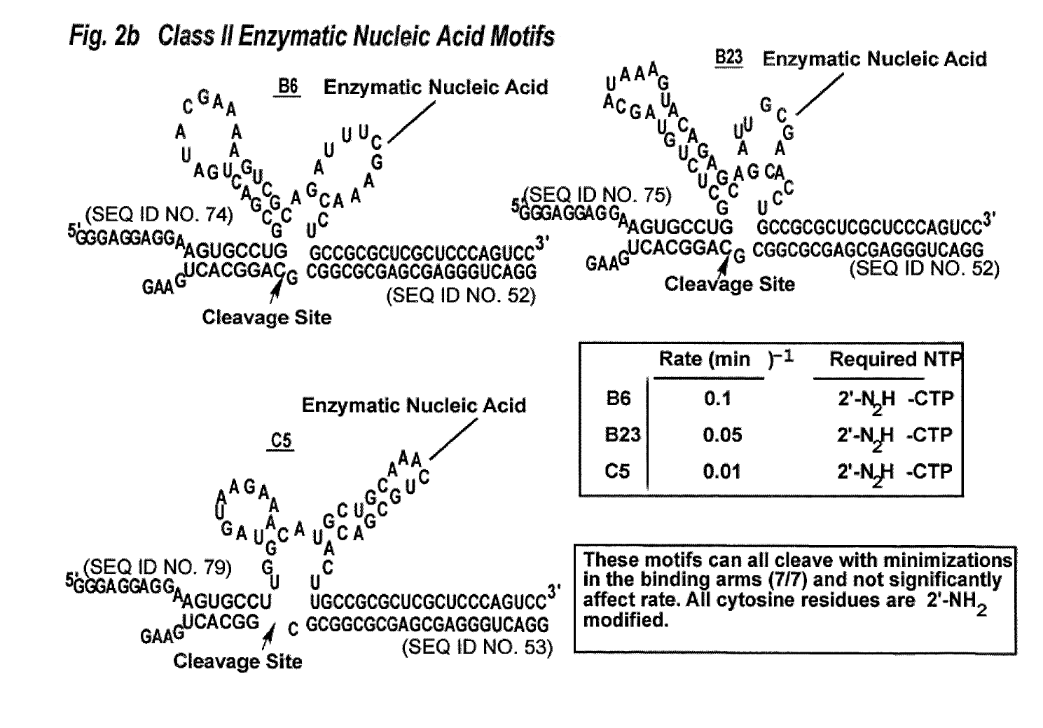

The sequence alignments shown in Table 1 can be divided into six primary groups (FIG. 2) and a few unique sequences. Secondary structure predictions of all sequences were made using Mfold (Jaeger et al., 1990, Methods in Enzymology, 182, 281-306; Zuker et al, 1991, Nucleic Acids Research, 19, 2702-2714) and are shown in FIG. 2. This analysis suggested two primary classes, one from each of the pools A and B, which are themselves related in structure. Inspection shows the A1 and A5 groups share a 13 base identity in a very similar predicted structure. The B2, B6 and B23 groups possess a 14 base loop with high sequence similarity. Furthermore, they share similar predicted structures to the A group. The most prominent variability seen in the reselected motifs falls in the 5′ half of the randomized region and is predicted to be a stem in both the A and B classes. The C pool yielded two motifs with the lowest observed rates and provided no reselected variants for comparison. Both C motifs have predicted structures very different from A and B class motifs. These predicted structures aided in the design of truncate variants to minimize the motif size as discussed below.

Activity With Physiological Divalent Metal Ion Concentrations

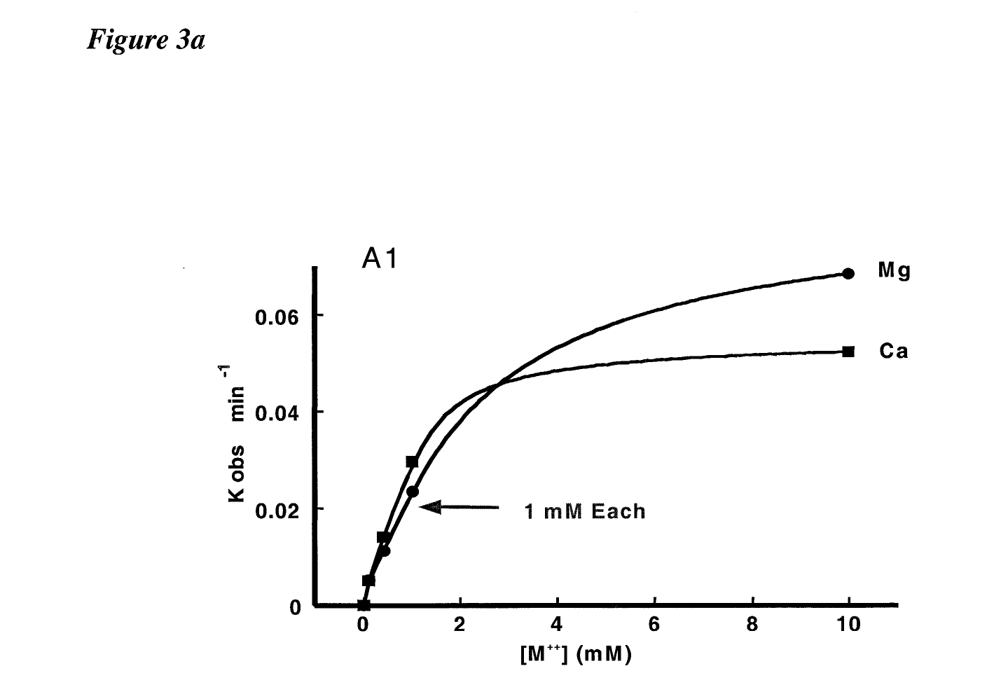

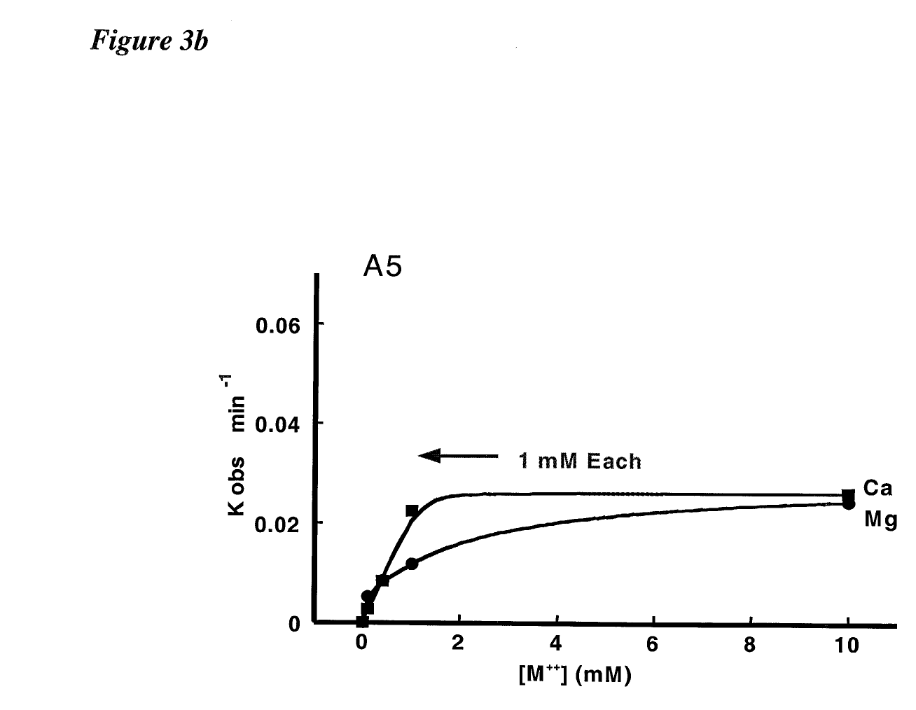

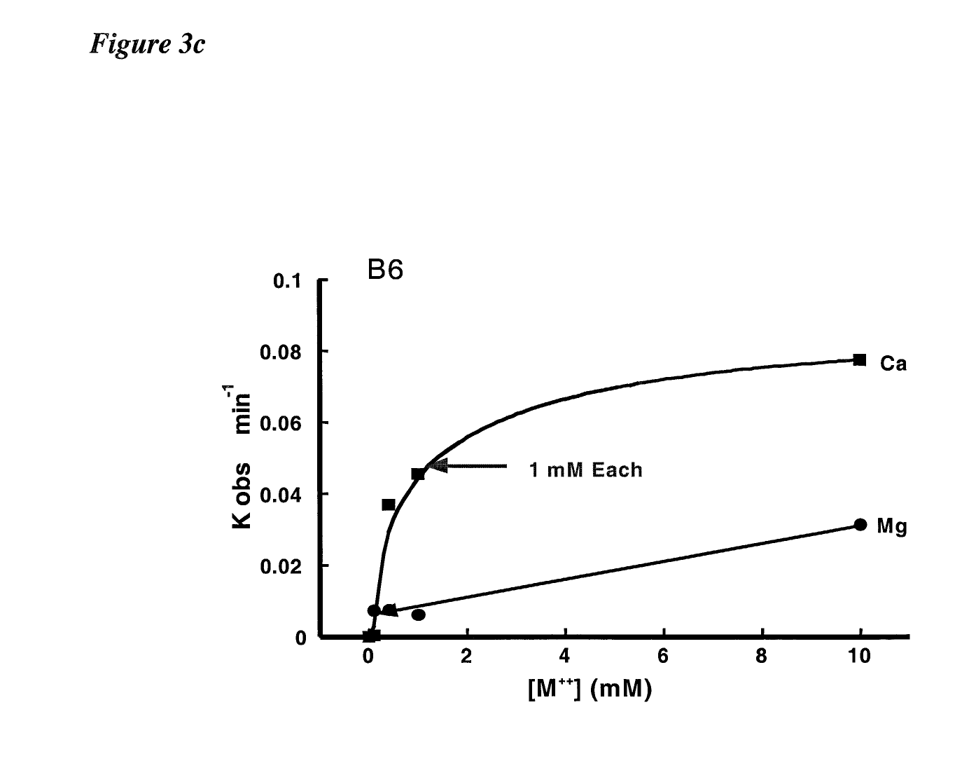

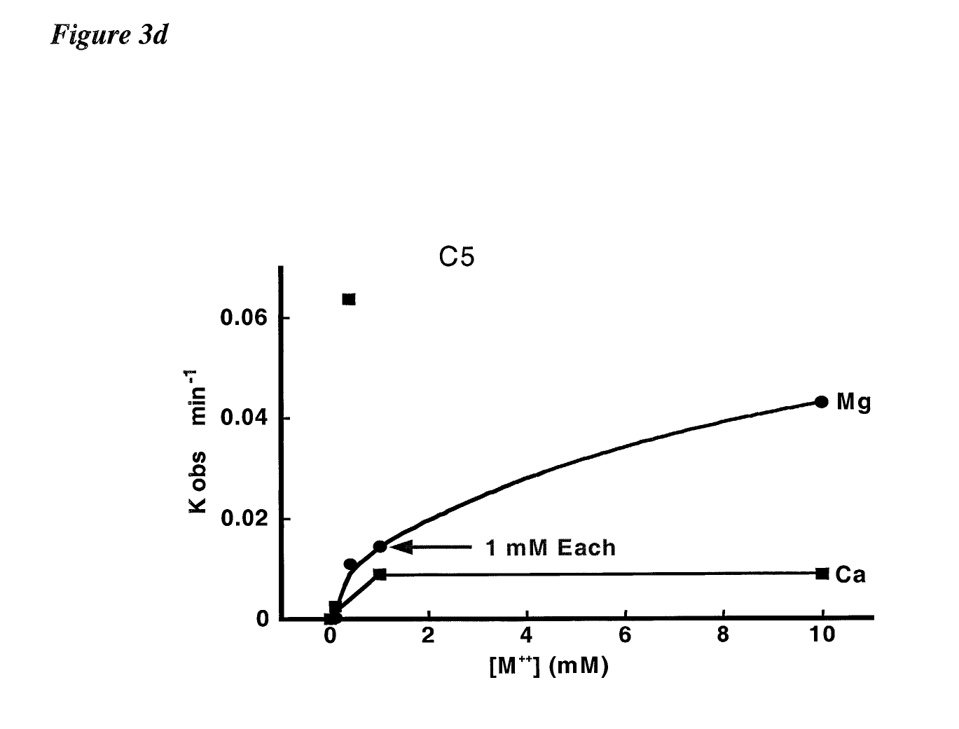

FIG. 3 shows the dependence of the single turnover cleavage rate on the concentration of magnesium and calcium. All motifs are able to function well in and beyond the physiological concentration range of either divalent metal ion. It is interesting to note that differing dependencies arose from the same selection; of particular interest is the A1 and A5 motifs. These two motifs share an identical 13 nucleotide loop, presumed to be the catalytic core, and differ only in the sequence of their stem loops. This difference in the stem sequence apparently accounts for either an enhanced catalytic rate as a function of increasing divalent ions, as seen in the A1 motif, or a catalytic rate insensitive to increasing divalent ion concentrations, as seen in the A5 motif. Thus the A class motif can be modulated in its response to divalent metal ions by the choice of sequence in its stem loop.

### Example 2

### Improving Nuclease Resistance and Ease of Synthesis

Modified NTP Requirements

In an effort to assess which of the modified NTPs were required for catalytic activity, separate transcriptions were carried out in which one of the modified NTPs was replaced by its corresponding ribonucleotidetriphosphate in the following parental motifs: A1, A5, B2, B6, B23 and C5. Cleavage activity assays indicated the 2′-NH₂-C was required for activity in all of these motifs. Initially, only C5 required the 2′-F-5-[(N-imidazole-4-acetyl) propylamine]-UTP, all other motifs showed no change in kinetic behavior in its absence. Interestingly, the C5 motif possessed the lowest cleavage rate measured. Furthermore C5 and its variants were eliminated during reselection. While it was hoped the imidazole functionality would allow the identification of superior catalysts, this was not the case. The one motif utilizing this modification proved ineffective when placed in a head-to-head reselection with the other catalysts. Moreover, this finding is corroborated by results of a parallel selection attempting to identify catalysts that allowed the imidazole moiety to function as a catalyst in the absence of divalent cations. In this experiment, conditions were identical to the B selection with the exception of the elimination of magnesium and calcium ions. No activity emerged from these pools despite permissive reaction conditions (20-30 minutes at 37° C.); selection was discontinued after the sixteenth round. While this is a negative result, it is consistent with the finding that winners from pools with the imidazole functionality present found no selective advantage in its use.

Size Reduction

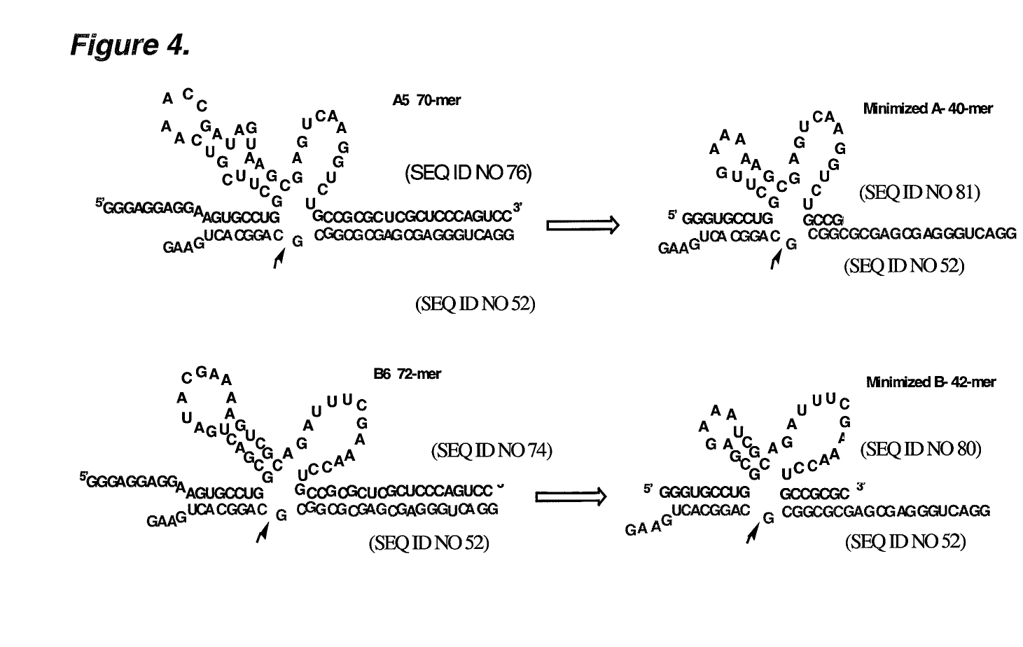

The minimum size requirements for the A and B enzymatic nucleic acid motifs were investigated in order to better understand their structural requirements, and to prepare for future large-scale synthesis of these enzymatic nucleic acids; in which smaller size translates into lower cost. Secondary structure predictions along with observed compensatory base changes in the stem regions were used to design the minimal structures shown in FIG. 4. These were then made enzymatically, but low RNA yields were obtained due to inefficient T7 promoter sequences in these transcripts. This in turn prevented cleavage assays from being carried out at the typical 400 nM catalyst concentration. However, trace amounts of the minimized A and B truncates showed 10 and 20% cleavage of substrate at on overnight time point (compared to <0.5% for substrate in the absence of enzymatic nucleic acid molecules). This strongly supported the predicted structures, and encouraged the pursuit of further synthetic truncations. These synthetic truncates were tested with concomitant attempts to reduce the 2° F. content and are shown in Table 2.

Reduction of 2′-F Content

Table 2 summarizes the impact on the catalytic rate of variants designed to test these substitutions and further investigates truncation in both the catalytic domain and the substrate binding arms. The sequences labeled 1-8 showed the progression from the variant most similar to the parental length and binding arm configuration to the most truncated motif with complete substitution of 2′-OMe's for 2′-F's. Sequence 8 also demonstrated that a more stable base pairing in the predicted stem structure improved the in vitro rate. As compared to the B motif sequences 9-14, the A-motif was more amenable to truncation and synthesis with 2′-OMe's replacing 2′-F's. While the B-motifs were still functional they were seriously compromised with regard to activity. Whether that was a function of arm length, 2′ substations or both was not assessed. The smallest A motif was a 36-mer and was slightly more active than its full-length parent. This prompted further development of the sequence 8 motif.

Reduction of 2′-NH2 Content

2′-NH₂-C nucleotides were systematically replaced with 2′-OMe nucleotides. Table 3 shows the variants tested and their activity values; sequence 8.1 is equivalent to the optimized sequence 8 (Table 2). The first 2′-NH₂ reduction was based on the high rate observed for sequence 5 (Table 2) that had a deletion in this 2′-NH₂-C position. The remaining NH₂ reduction enzymatic nucleic acids are a systematic screen of possible combinations of two 2′-NH₂-C's or single 2′-NH₂-C positions. With only two 2′-NH₂-C's a modest 3-fold loss in activity was observed, and activity can still be detected with only one position retaining the 2′-NH₂ modification. Applicant proceeded with the sequence containing two 2′-NH₂-C's as shown in sequence 8.3.

Protection From Exonucleases and Serum Stability

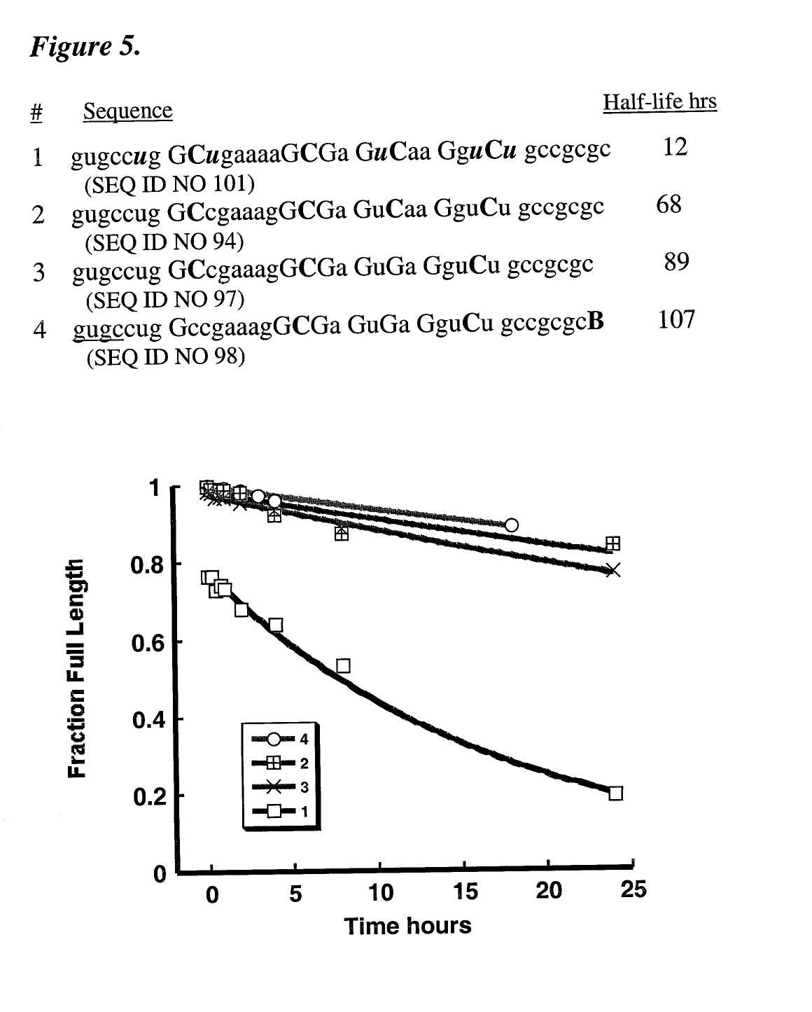

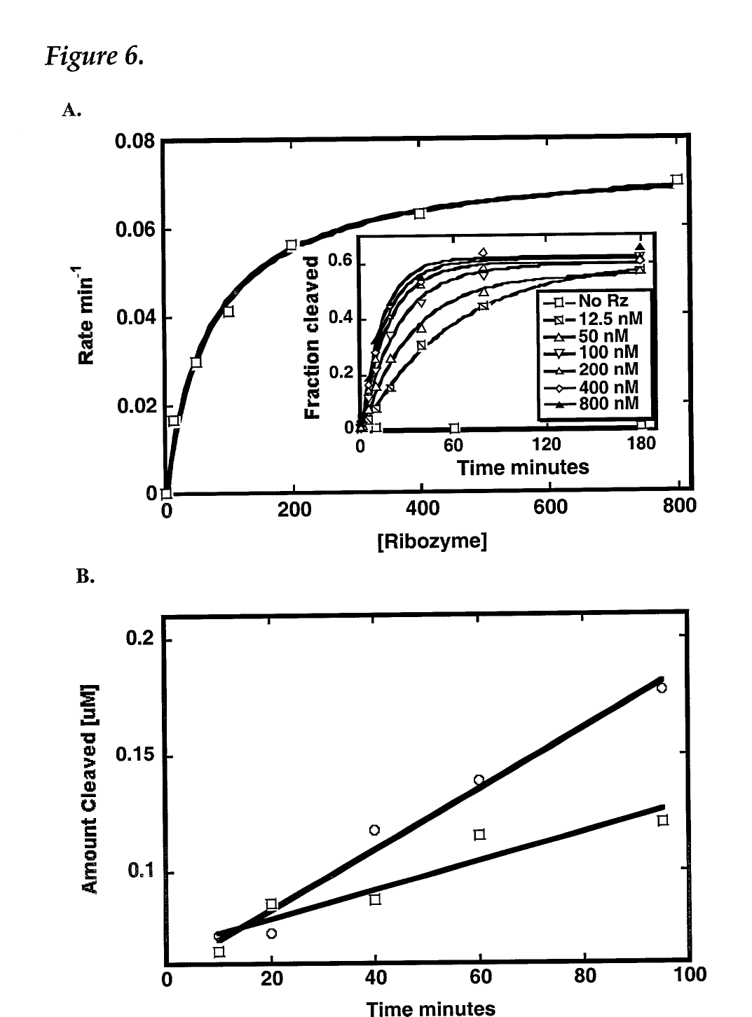

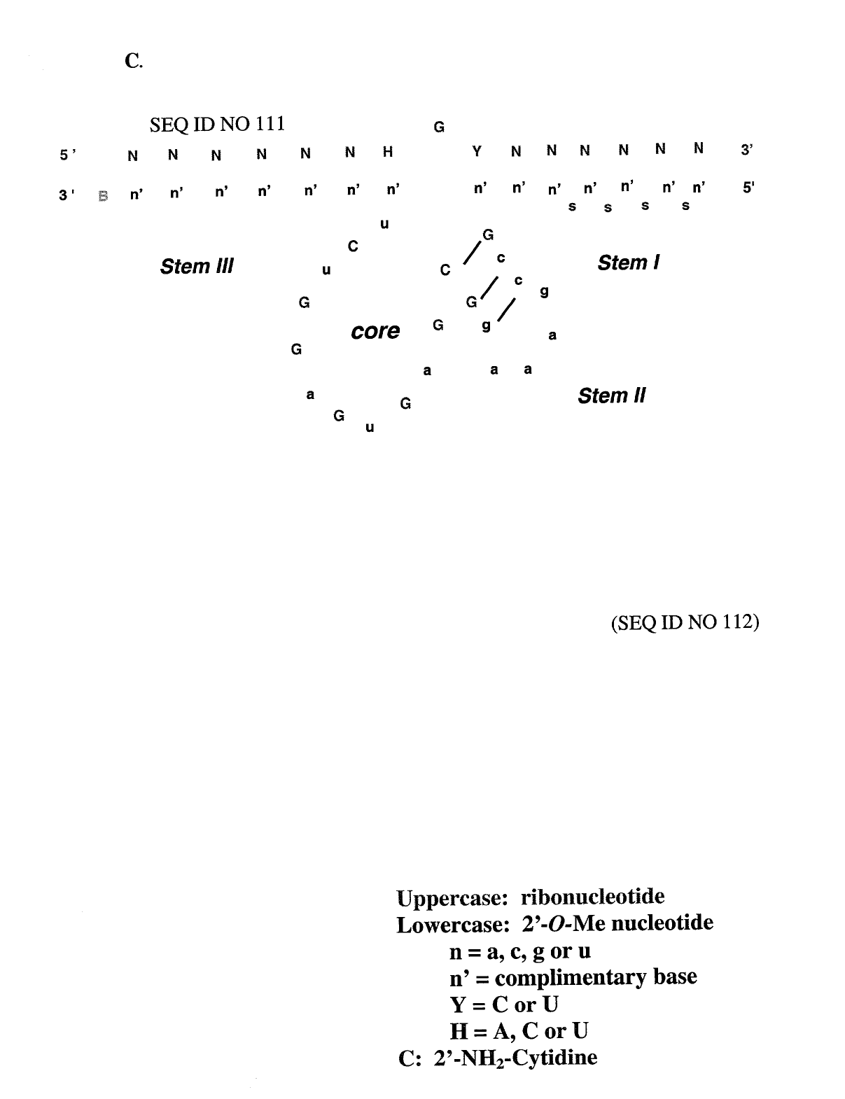

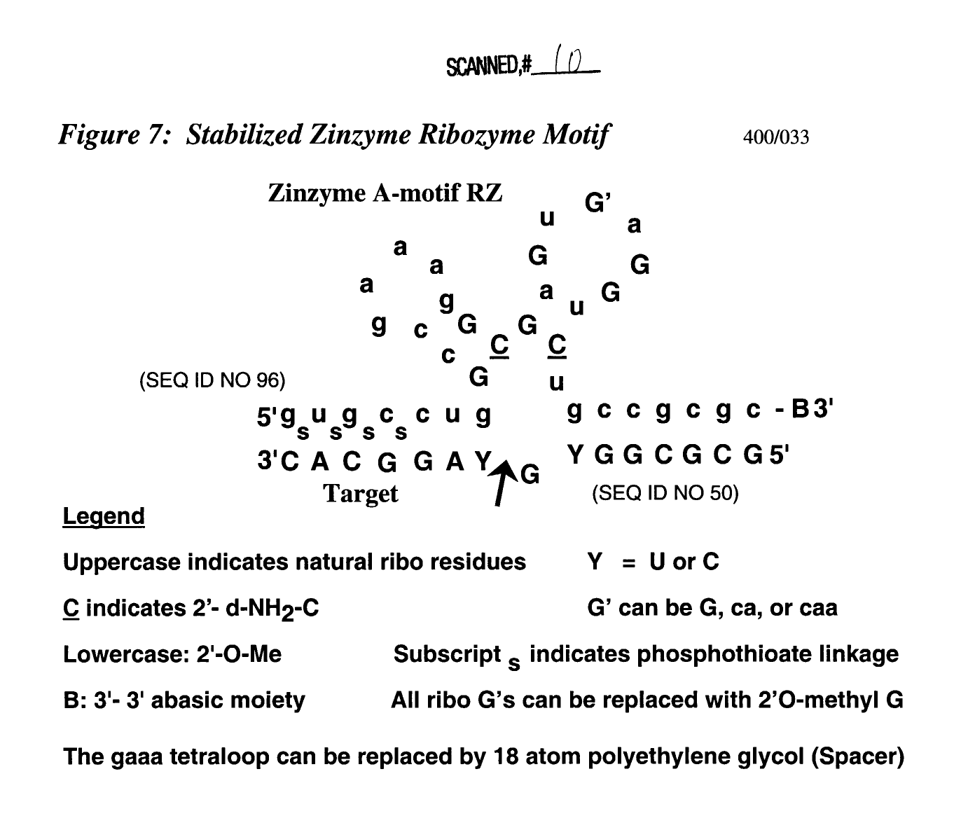

The stability of an oligonucleotide in a biological environment is critical to it's utility as a therapeutic. To assess this, the lifetime in human serum was measured for two variants of the 8.3 motif. One variant maintained the 2′-F modification at the U positions while the other maintained all U positions as 2′-OMe's; (see FIG. 5). This generated a >7-fold improvement in stability; t_{½}=90 hours. While this appeared to be sufficiently stable a further improvement was made based on the stabilization efforts of the hammerhead motif. It had been demonstrated previously that modification of the binding arms with four phosphorothioate positions in the 5′ terminus and in inverted abasic residue on the 3′ terminus greatly limits degradation by exonucleases. These same modifications were added to this new motif with no effect on the observed in vitro cleavage rates (FIG. 6) and provided a slight increase in the stability in human serum (FIG. 5).

### Example 3

### Substrate Sequence Requirements for Cleavage

To take full advantage of a novel motif in high throughput RNA target site identification it was necessary to determine the substrate requirement rules for activity. The 8.3-motif (Table 3) was chosen. All possible nucleotides were tested in the bulged position with the wild type binding arms. Minimal cleavage (<5%) was seen with overnight incubation in PBM at 37° C. and these substrates were assessed no further. While maintaining G in the bulge position all sixteen possible combinations of flanking nucleotides were assessed with canonical pairings of enzymatic nucleic acid to substrates. Table 4 shows the fraction cleaved at 2 and 14 hour time points in PBM at 37° C. The most active matched eznymatic nucleic acid-substrates have a pyrimidine 5′ to the bulged G and C, U or A 3′ to the bulged G. Therefore the recognition rules for the most active constructs are 5′-Y—G—H-3′ (where Y is C or U, and H is A, C or U). However, the motif still demonstrated cleavage with reduced activity when purines were 5′ to the bulged G and when C, U or A was 3′ to the bulged G; of exception is 5′-GGA-3'substrate. Furthermore, the motif has demonstrated very similar cleavage activity on numerous substrates with the 5′-Y—G—H-3′ rule and all other binding arm positions different from the K-ras sequence used in the selection. Thus, this motif provides a novel set of minimally restrictive substrate sequence requirements that allows access to cleavage sites not possible with the current stabilized hammerhead motif.

Kinetics

A minimal assessment of the kinetic characteristics was made to better understand the catalytic activity in a physiological setting. Single and multi-turnover results for the optimized motif (motif 8.3 with terminal phosphorothioates and a 3′-inverted abasic cap) are is shown in FIG. 6 along with its final chemical format. Single turnover measurements demonstrated this motif displays a k_{cat}=0.07 min⁻¹ and a Kₘ=70 nM; k_{cat}/Kₘ=10⁶ M⁻¹ min⁻¹. Multiple turnover assays performed at two different enzymatic nucleic acid concentrations yielded a rate of 0.013 min⁻¹. The turnover rate was ˜3-fold slower than k_{cat}, suggesting either substrate association or product release was rate limiting.

To more accurately reflect the monovalent environment within cells, a single turnover measurement was made in PBM with the Potassium concentration raised from 14 to 140 mM. This change resulted in a very modest, 1.5-fold reduction in the cleavage rate. Thus, this motif demonstrated good catalytic activity in conditions simulating the physiological conditions within which a therapeutic agent would be required to operate.

Taken together, these results have demonstrated the ability of in vitro selection to identify an enzymatic nucleic acid predisposed for use as a drug. By optimizing the best of the selected catalysts a novel motif was developed, designated the Zinzyme™, FIG. 6. Zinzyme possesses good catalytic activity in physiological conditions with non-restrictive substrate recognition rules. It is nuclease resistant and small in size, thereby demonstrating those properties desired in a potential therapeutic.

### Example 4

### Library/Pool Preparation

Template oligonucleotide was converted to double-stranded (d.s.) DNA; 5 nmol template=5′-TGG GAG CGA GCG CGG CA N₃₅ AGG CAT ATT TAT ATC CTA TAG TGA GT-3′ (SEQ ID NO: 120), 10 nmol Primer 1 (R.T.) 5′-TGG GAG CGA GCG CGG CA-3′ (SEQ ID NO: 119) and 5 nmol Primer 2 (T7 promoter) 5′-TAA TAC GAC TCA CTA TAG GAT ATA AAT ATG CCT-3′ (SEQ ID NO: 118) were run through two PCR cycles. Product was twice ethanol precipitated and twice passed through NAP-5 G-25 resin (Amersham-Pharmacia biotech). Product size was verified on 8% native PAGE. Initial pools were transcribed using the mutant T7 Y639F RNA polymerase using the following final conditions: 40 mM Tris-HCl pH 8.5, 16 mM MgCl₂, 5 mM DTT, 1 mM Spermidine, 0.02% Triton X-100, 1 mM LiCl, 5% polyethyleneglycol 8000, 1 mM each NTP, trace [α-³²P]-GTP, 1 nmol d.s. DNA template (0.5 μM final concentration). The following NTP mixes were used (see FIG. 1): Selection A -2′-F-ATP, 2′-NH₂-CTP, GTP and 2′-F-UTP; Selections B and C -2′-F-ATP, 2′-NH₂-CTP, GTP and 2′-F-5-[(N-imidazole-4-acetyl) propylamine]-UTP. Modified NTP's were prepared as described (Matulic-Adamic et al., 2000, Bioorganic and Medicinal Chemistry, 10, 1299-1302). The reaction proceeded 3-6 hours at 37° C. RNA product was purified as described above for oligonucleotides. Specific activity of the [α-³²P]-GTP was used to calculate the concentration of RNA product, assuming a 25% G content. Conditions with these nucleotide complements generated an apparent average of 10 copies of RNA per DNA template.

Template oligonucleotides used for reselection are shown where numbers indicate the NTP mixtures giving a 36% level of random incorporation of the primary NTP listed: 5=G, 6=A 7=C8=T. A1-5′-GGA CTG GGA GCG AGC GCG GCA (SEQ ID NO: 51)-65 778 86578 76775 75556 65555 87785 86577-AGG CAC TTC CTT CCT CCC TAT AGT GAG TCG TAT TA-3′ (SEQ ID NO: 18); A5-5′-GGA CTG GGA GCG AGC GCG GCA (SEQ ID NO: 51)-65778 86578 76788 57868 76688 86576 55677-AGGCA CTTCC TTCCT CCCTA TAGTG AGTCG TATTA-3′ (SEQ ID NO: 18); B2-5′-GGA CTG GGA GCG AGC GCG GCA (SEQ ID NO: 51) -65778 66888 77676 87867 77868 58568 85756 6577-AGG CAC TTC CTT CCT CCC TAT AGT GAG TCG TAT TA-3′ (SEQ ID NO: 18); B6-5′-GGA CTG GGA GCG AGC GCG GCA (SEQ ID NO: 51)-66888 76555 87867 65788 88768 58756 87677-AGG CAC TTC CTT CCT CCC TAT AGT GAG TCG TAT TA-3′ (SEQ ID NO: 18); B23-5′-GGA CTG GGA GCG AGC GCG GCA (SEQ ID NO: 51)-66868 76755 87867 87578 88586 78656 77-AGG CAC TTC CTT CCT CCC TAT AGT GAG TCG TAT TA-3′ (SEQ ID NO: 18); C5-5′-GGA CTG GGA GCG AGC GCG GCA (SEQ ID NO: 51)-56868 76756 88867 56758 68887 88578 5775-AGG CAC TTC CTT CCT CCC TAT AGT GAG TCG TAT TA-3′ (SEQ ID NO: 18). All mutagenized pools were prepared as described above.

### Example 5

### Column Preparation

Resin 1-Substrate oligonucleotide with end terminus coupling moieties [5′-b-L-GGA CUG GGA GCG AGC GCG GCG CAG GCA CUG AAG-L-S-B-3′; (SEQ ID NO: 19) where L=Spacer 18 (Glenn Research cat# 10-1918-nn), b=Biotin Phosphoramidite (Glenn Research cat# 10-1953-nn), B=inverted deoxy abasic resin, S=thiol-modifier C6 S—S (Glenn Research cat# 10-1936-nn)] was reduced to generate a 3′-thiol terminus as follows; 14 nmol substrate in 20 ul of 11.7 M B-mercaptoethanol was incubated for 150 minutes at 22° C. The substrate was twice passed through a MicroSpin G-25 column. This was then added to Ultralink-lodoacetyl (Pierce cat# 53155) equilibrated with 50 mM Tris-HCl pH 7.5, 5 mM EDTA and incubated with rocking at 22° C. for 90 minutes. The resin was washed with equilibration buffer and stored at 4° C. until use. Assessment of coupling efficiency indicated the final concentration of the resin bound substrate was approximately 2.0 μM. Resin 2-UltraLink Immobilized NeutrAvidin Plus (Pierce cat# 53151) was equilibrated in 20 mM HEPES pH 7.4, 2.0 M NaCl and stored at 4° C. until use.

### Example 6

### Selection

Initial pools (0.8 nmoles pool A, 1.65 nmoles pools B and C) were incubated with 2 μM substrate-Resin 1 (1 ml pool A, 2 ml for pools B and C) for 10 minutes at 22° C. followed by 10 minutes in 20 mM HEPES pH 7.4, 14 mM NaCl, 10 mM KCl (Physiological Buffer without metals =PB) at 37° C. for pools A and B, while pool C was maintained at 22° C. All subsequent steps were maintained at 37° C. for pools A and B and 22° C. for pool C. Resin was washed with 20 column volumes of PB. The reaction was triggered by the addition of PB plus 1 mM MgCl₂, 1 mM CaCl₂ (PB+Metals=PBM). Two column volumes of PBM were rapidly passed over the resin. The column was capped and incubated with 2 column volumes of PBM for 20 minutes. Subsequently the resin was washed with 5 column volumes of PBM. All PBM fractions were pooled and brought to a final NaCl concentration of 2 M. 2001L of NeutraAvidin-Resin 2 was added and allowed to incubate with rocking at 22° C. The resin was washed with 10 ml of 20 mM HEPES pH 7.4, 2 M NaCl. Then 400 μL of 10 M Urea, 1 M NaCl (Urea Buffer=UB) was rapidly (<10 seconds) passing over the resin. The column was capped and sealed with 1 mL UB and incubated at 75° C. The eluant and an additional 500 μL UB wash were pooled with the initial UB wash. The RNA was ethanol precipitated using 100 μg glycogen as carrier and resuspended in 100 μL of sterile water. Standard RT-PCR provides the d.s. DNA for the subsequent generation.

### Example 7

### Cloning and Sequencing

Pools were cloned using Novagen's Perfectly Blunt Cloning kit (pT7Blue-3 vector) following the kit protocol. Clones were screened for insert by PCR amplification using vector-specific primers. Positive clones were sequenced using ABI Prism 7700 sequence detection system and vector-specific primer. Sequences were aligned using MacVector software; two-dimensional folding was done using Mulfold software [46]. Individual clone transcription units were constructed by PCR amplification with 50 pmol each primer 1 and primer 2 in 1× PCR buffer, 0.2 mM each DNTP, and 2.5 U of taq polymerase in 100 μl volume cycled as follows: 95° C., 4 min; (95° C., 30 sec; 54° C., 30 sec; 72° C., 1 min)×20. Transcription units were ethanol precipitated, rehydrated in 30 μl H₂O , and 10 μl was transcribed in 100 μl volume and purified as previously described.

### Example 8

### Kinetic Analysis

Single turnover kinetics were performed with trace amounts of 5′-³²P-labeled substrate and 500 nM pool or enzymatic nucleic acid in PBM at 37° C. The sequence of the substrate used is 5′-GCGCGGCGCAGGCAC-3′ (SEQ ID NO: 6). Substrate (2×) in PBM and 2× enzymatic nucleic acid in PBM were incubated separately at 70° C. for 1 min followed by equilibration to 37° C. for at least 3 min. An equal volume of substrate and enzymatic nucleic acid or pool was mixed and the reaction was incubated at 37° C. Samples were quenched in >2 volume 8 M Urea, 50 mM EDTA, 1× TBE and 0.04% Xylene Cynol at various time points. Samples were heated to 90° C. for 1 min prior to separation on 15% denaturing sequencing gels. Gels were imaged using a phosphorimager and quantified using ImageQuant software (Molecular Dynamics). Curves were fit to a single exponential. Unless stated explicitly all rates reported are given in 1× PBM at 37° C. Single timepoints cleavage as seen in the substrate rule cleavage experiments was preformed with enzymatic nucleic acid in excess as above. Multiple turnover kinetics were performed in condition as described above however the final concentration of enzymatic nucleic acid was either 50 or 100 nM while the substrate was 2 μM. The method of initial rates was used to determine the rate constant.

### Example 9

### Stability Analysis

100 pmoles of enzymatic nucleic acid was 5′-end labeled by a standard kinase protocol and added to 500 pmoles of unlabeled enzymatic nucleic acid. The mixture was dried and resuspended in 20 μl of fresh human serum. The suspension was incubated at 37° C. and aliquants (2μL) were removed at 0, 0.25, 0.5, 0.75, 1, 2, 4, 8 and 24 hours and quenched in 95% formamide, 20 mM EDTA, 0.05 xylene cynol on ice. Samples were analyzed by denaturing 20% PAGE. Degradation was quantitated using a Molecular Dynamics Phosphorimager. Half-lives were estimated by fitting the fraction of full length enzymatic nucleic acid remaining as a function of time, to a single exponential equation extrapolated to complete degradation.

### Example 10

### Reduction of Ribose Residues in Zinzyme Nucleic Acid Catalysts

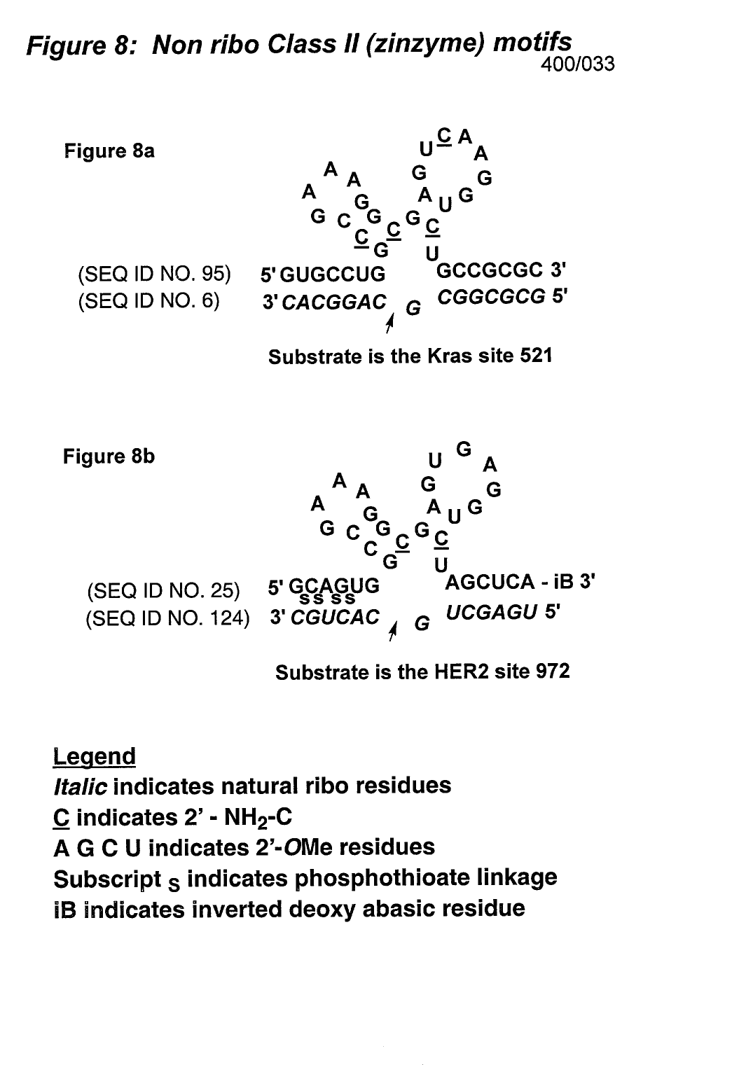

Zinzyme nucleic acid catalysts were tested for their activity as a function of ribonucleotide content. A Zinzyme having no ribonucleotide residue (ie., no 2′-OH group at the 2′ position of the nucleotide sugar) against the K-Ras site 521 was designed. These molecules were tested utilizing the chemistry shown in FIG. 8a. The in vitro catalytic activity of the zinzyme construct was not significantly effected (the cleavage rate reduced only 10 fold).

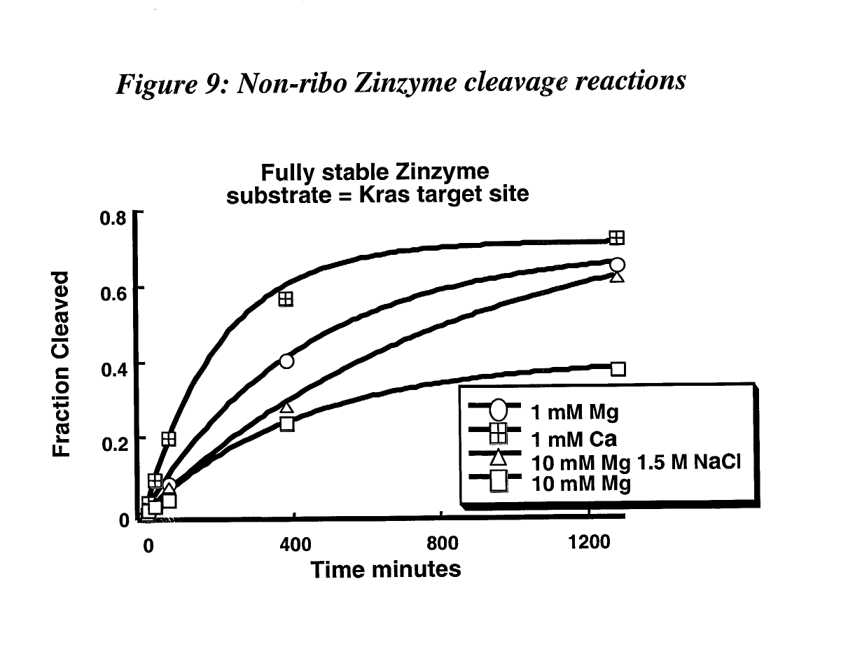

The Kras zinzyme shown in FIG. 8a was tested in physiological buffer with the divalent concentrations as indicated in the legend (high NaCl is an altered monovalent condition shown) of FIG. 9. The 1 mM Ca⁺⁺ condition yielded a rate of 0.005 min⁻ while the 1 mM Mg⁺⁺ condition yielded a rate of 0.002 min⁻. The ribose containing wild type yields a rate of 0.05 min⁻ while substrate in the absence of zinzyme demonstrates less than 2% degradation at the longest time point under reaction conditions shown. This illustrates a well-behaved cleavage reaction catalyzed by a non-ribose containing catalyst with only a 10-fold reduced cleavage as compared to ribonucleotide-containing zinzyme and vastly above non-catalyzed degradation.

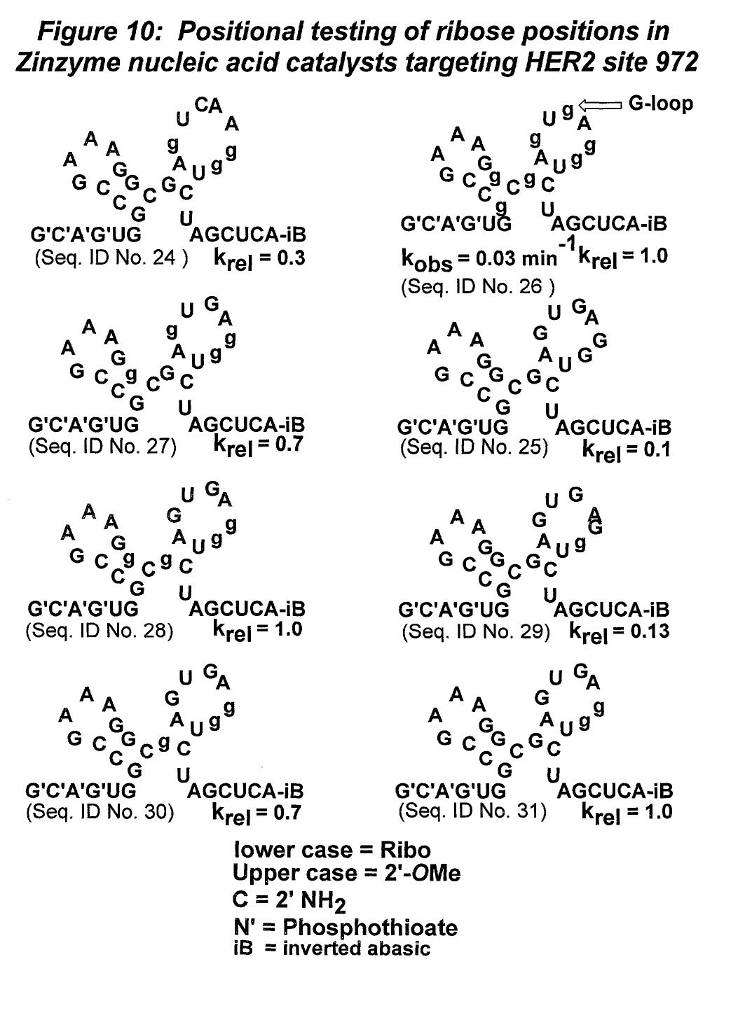

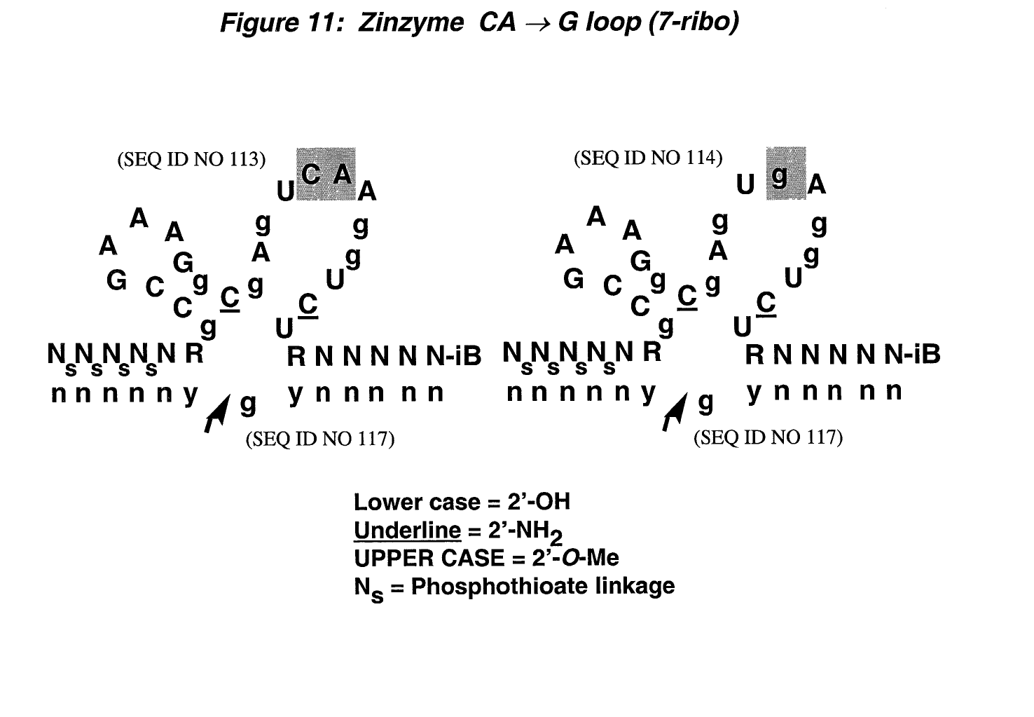

A more detailed investigation into the role of ribose positions in the Zinzyme motif was carried out in the context of the HER2 site 972 (Applicant has further designed a fully modified Zinzyme as shown in FIG. 8b targeting the HER2 RNA site 972). FIG. 10 is a diagram of the alternate formats tested and their relative rates of catalysis. The effect of substitution of ribose G for the 2′-O-methyl C-2′-O-methyl A in the loop of Zinzyme (see FIG. 11) was insignificant when assayed with the Kras target but showed a modest rate enhancement in the HER2 assays. The activity of all Zinzyme motifs, including the fully stabilized “0 ribose” (RPI 19727) are well above background noise level degradation. Zinzyme with only two ribose positions (RPI 19293) are sufficient to restore “wild-type” activity. Motifs containing 3 (RPI 19729), 4 (RPI 19730) or 5 ribose (RPI 19731) positions demonstrated a greater extent of cleavage and profiles almost identical to the 2 ribose motif. Applicant has thus demonstrated that a Zinzyme with no ribonucleotides present at any position can catalyze efficient RNA cleavage activity. Thus, Zinzyme enzymatic nucleic acid molecules do not require the presence of 2′-OH group within the molecule for catalytic activity.

### Example 11

### Zinzyme Tetraloop Combinatorial Study

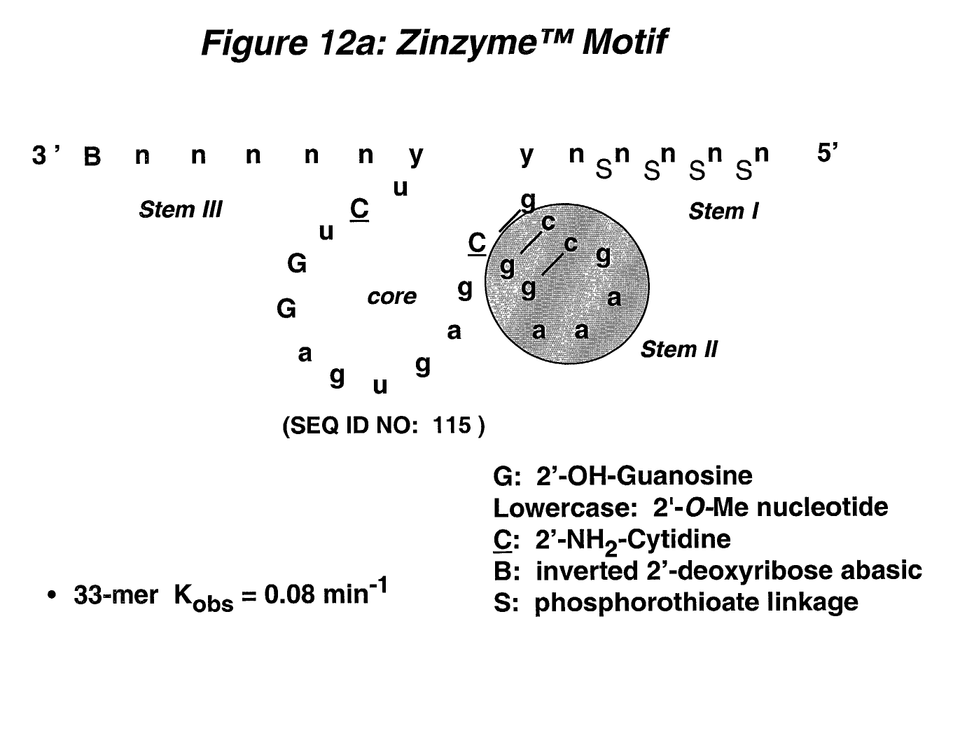

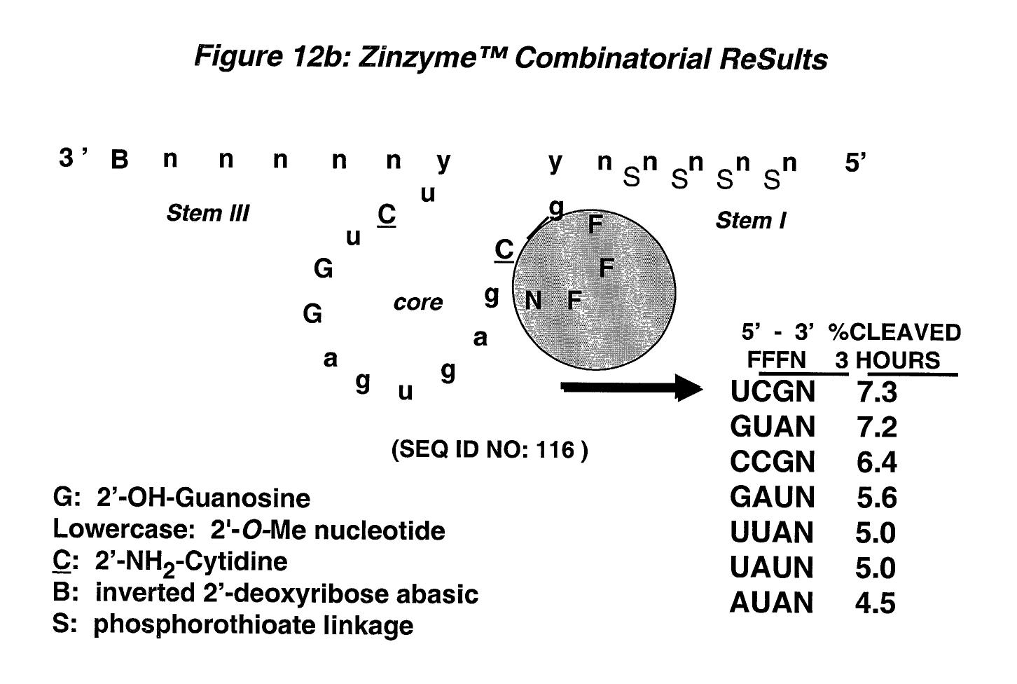

A combinatorial study of differing Zinzyme tetraloop sequences was performed wherein all possible four-nucleotide combinations that can replace the Zinzyme stem II were tested for cleavage activity (see FIG. 12). A single three hour time point was used for assensing cleavage. Seven initial pools gave signal from the tetra-loop combinatorial screen. Results are summarized in Table 5

The UUAA tetraloop was known to be an active Zinzyme (K=0.0005), and served as a positive control. All twenty-eight Zinzymes from the active pools were tested doing a full time course to obtain rate constants. Of the twenty-eight tested, seven ribozymes showed cleavage signals that were strong enough to obtain rates. Each of these seven Zinzymes represents one of the original active pools (one Zinzyme from each pool that gave signal.) This means the combinatorial assay was effective in picking active pools. The UUAA substituted Zinzyme shows the greatest activity.

### Example 12

### Zinzyme Modification Study

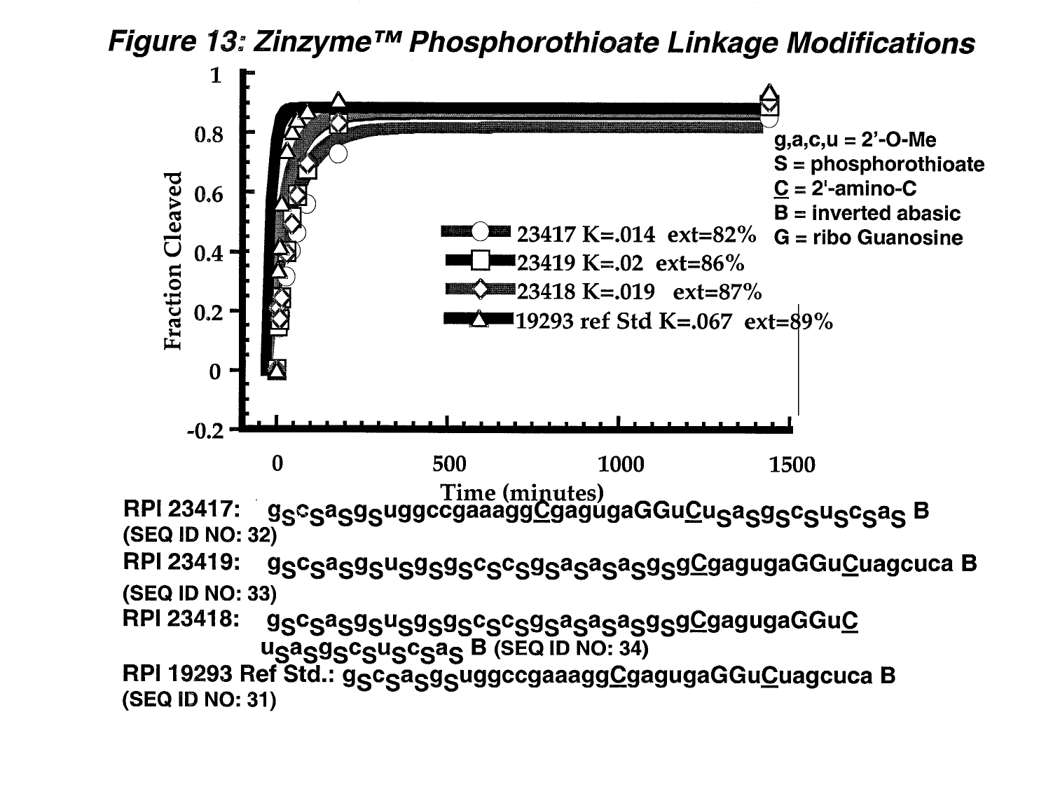

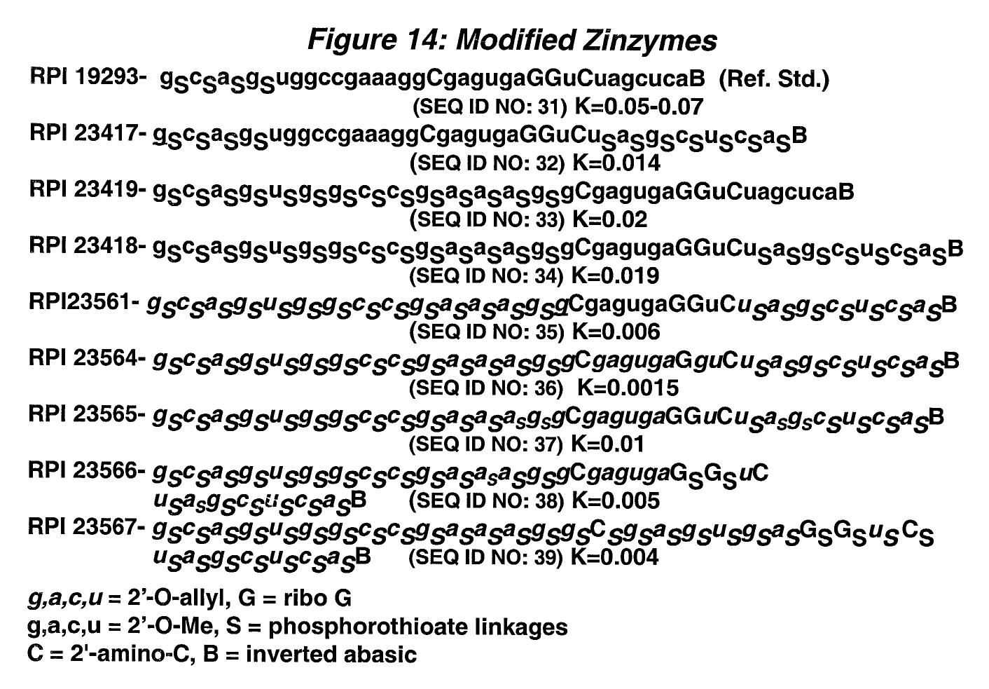

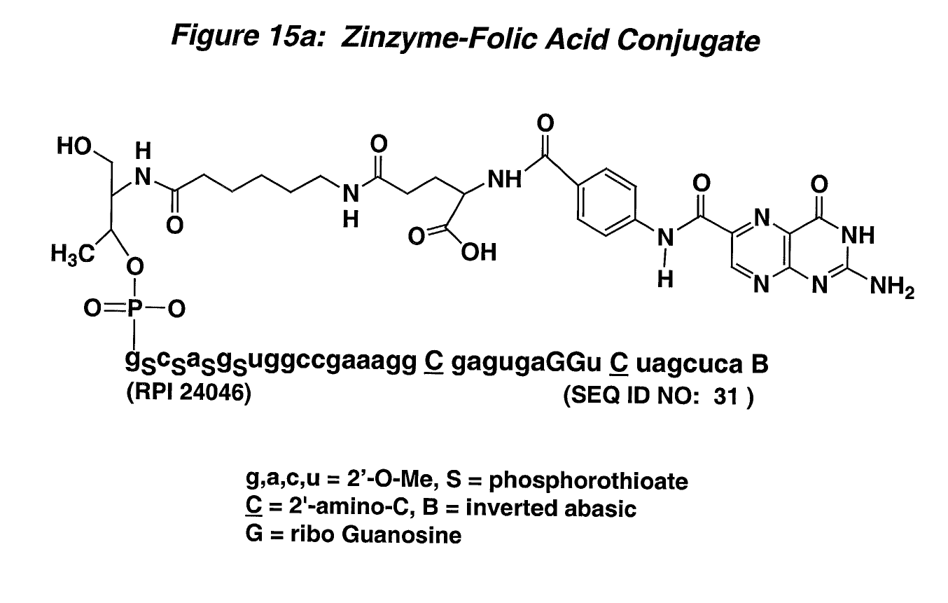

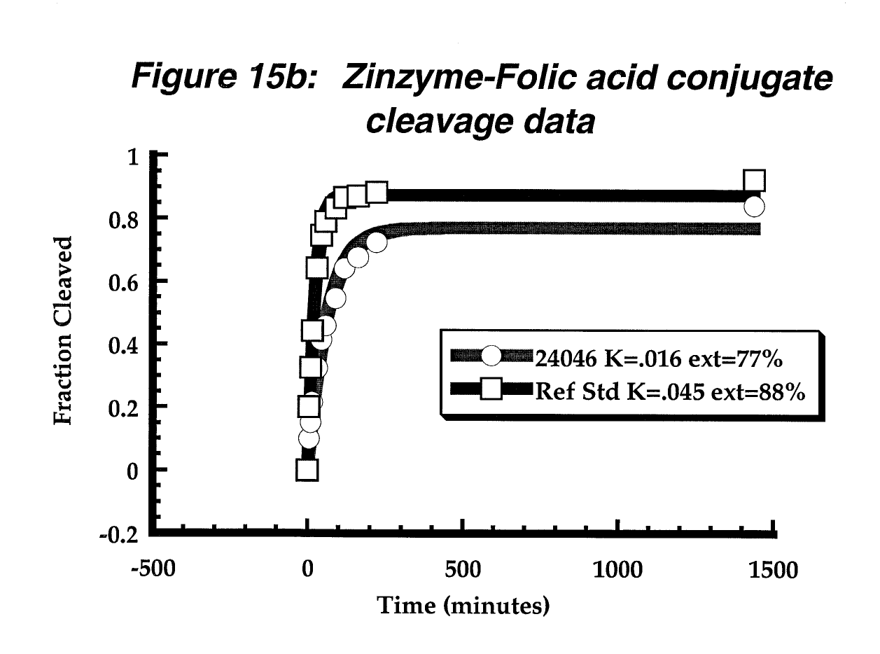

Various modifications can be introduced into the Zinzyme motif in order to modulate, for example, Zinzyme stability and pharmacokinetics. Zinzymes with varying phosphorothioate intemucleotide linkage substitutions were synthesized and tested for cleavage activity using the methods described herein. The Zinzyme sequences and cleavage data are shown in FIG. 13. Zinzymes with 2′-O-allyl modifications were synthesized and tested for cleavage activity using the methods described herein. FIG. 14 shows the Zinzyme sequences and cleavage data for this study. In addition, a 5′-folic acid Zinzyme conjugate was prepared and tested for cleavage activity (FIG. 15). Diagnostic uses Enzymatic nucleic acid molecules of this invention can be used as diagnostic tools to examine genetic drift and mutations within diseased cells or to detect the presence of specific RNA in a cell. The close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes that are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, radiation or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with related conditions. Such RNA is detected by determining the presence of a cleavage product after treatment with a enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild type and mutant RNAs in the sample population. Thus, each analysis involves two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of MRNA whose protein product is implicated in the development of the phenotype is adequate to establish risk. If probes of comparable specific activity are used for both transcripts, then a qualitative comparison of RNA levels is adequate and will decrease the cost of the initial diagnosis. Higher mutant form to wild-type ratios are correlated with higher risk whether RNA levels are compared qualitatively or quantitatively.

Additional Uses

Potential usefulness of sequence-specific enzymatic nucleic acid molecules of the instant invention has many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to down-regulate gene expression of target genes in bacterial, microbial, fungal, viral, and eukaryotic systems including plant, or mammalian cells.

[image]

[image]

Underlined sequences indicate primary selection sequences, non-underlined sequences indicate reselection isolates. Shaded box indicates area of high similarity. Rates are derived from single turnover reactions with product formation as a function of time fit to a first order exponential.

[image]

TABLE 3
2′-deoxy-2′-amino Cytidine Requirements for Zinzyme Activity.

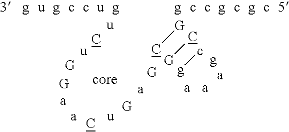

(SEQ ID NO: 21)

| | SEQ ID | | % cleaved at |
|---|---|---|---|
| Core sequence 5′ → 3′ | No: | k_{cat} mm⁻¹ | 3 hours |
| 8.1 GCcgaaagGCGaGuCaaGGuCu | 40 | 0.070 | 80 |
| 8.2 GCcgaaagGCGaGucaaGGuCu | 41 | 0.030 | 70 |
| 8.3 GccgaaagGCGaGucaaoGuCu | 42 | 0.024 | 70 |
| 8.4 GCcgaaagGCGaGucaaGGucu | 43 | ND | 10 |
| 8.5 GCcgaaagGcGaGucaaGGuCu | 44 | 0.00 | 0 |
| 8.6 GccgaaagGcGaGucaaGGuCu | 45 | 0.00 | 0 |
| 8.7 GccgaaagGCGaGucaaGGucu | 46 | ND | 10 |
| 8.8 GCcgaaagGcGaGucaaGGucu | 47 | ND | ND |

TABLE 4
Substrate recognition and cleavage

| | | % | % |
|---|---|---|---|
| | SEQ | cleaved | cleaved |
| | ID | at 2 | at 14 |
| | No | Hours | Hours |
| 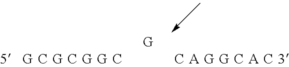 | 6 | | |
| 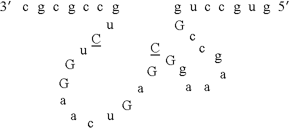 | 93 | | |
| 5′ GCGCGG AGA AGGCAC 3′ | 1 | 4 | 26 |
| 5′ GCGCGG AGC AGGCAC 3′ | 2 | 8 | 52 |
| 5′ GCGCGG AGG AGGCAC 3′ | 3 | 2 | 1 |
| 5′ GCGCGG AGU AGGCAC 3′ | 4 | 12 | 49 |
| 5′ GCGCGG CGA AGGCAC 3′ | 5 | 43 | 64 |
| 5′ GCGCGG CGC AGGCAC 3′ | 6 | 60 | 78 |
| 5′ GCGCGG CGG AGGCAC 3′ | 7 | 4 | 2 |
| 5′ GCGCGG CGU AGGCAC 3′ | 8 | 47 | 80 |
| 5′ GCGCGG GGA AGGCAC 3′ | 9 | <1 | 21 1 |
| 5′ GCGCGG GGC AGGCAC 3′ | 10 | 5 | 43 |
| 5′ GCGCGG GGG AGGCAC 3′ | 11 | 2 | 4 |
| 5′ GCGCGG GGU AGGCAC 3′ | 12 | 8 | 51 |
| 5′ GCGCGG UGA AGGCAC 3′ | 13 | 27 | 79 |
| 5′ GCGCGG UGC AGGCAC 3′ | 14 | 35 | 73 |
| 5′ GCGCGG UGG AGGCAC 3′ | 15 | <1 | <1 |
| 5′ GCGCGG UGU AGGCAC 3′ | 16 | 51 | 49 |

TABLE 5
Zinzyme tetraloop combinatorial results

| Pool sequence | % cleaved (3h) | Active Rz | k_{obs} |
|---|---|---|---|
| AUAN | 4.5 | AUAA | 0.0017 |
| GUAN | 7.2 | GUAA | 0.001 |
| UUAN | 5 | UUAA | 0.005 |
| CCGN | 6.4 | CCGG | 0.004 |
| GAUN | 5.6 | GAUC | 0.0033 |
| UCGN | 7.3 | UCGA | 0.0045 |
| UAUN | 5 | UAUA | 0.0011 |

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Thus, additional embodiments are within the scope of the invention and within the following claims.

We claim:

1. A compound having a Formula I: 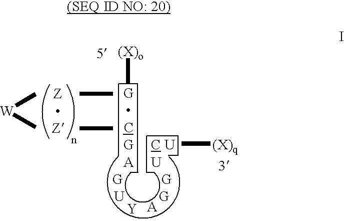
wherein each X, Y, and Z represents independently a nucleotide which can be the same or different; q is an integer greater than or equal to about 3; n is an integer from 0 to about 10; o is an integer greater than or equal to about 3; Z′ is a nucleotide complementary to Z; each X(q) and X(o) are oligonucleotides which are of sufficient length to stably interact independently with a target nucleic acid sequence; W is a linker of ≧about 2 nucleotides in length or can be a non-nucleotide linker; A, U, G, and C represent nucleotides; C is 2′-amino; and represents a chemical linkage.

2. The compound of claim 1, wherein the “q” in said enzymatic nucleic acid molecule is an integer of about 4, 5, 6,7, 8, 9, 10, 11, 12, or 15.

3. The compound of claim 1, wherein the “n” in said enzymatic nucleic acid molecule is an integer of about 0, 1, 2, 3, 4, 5, 6, or 7.

4. The compound of claim 1, wherein the “o” in said enzymatic nucleic acid molecule is an integer of about 3, 4, 5, 6, 7, 8, 9, 10, 11, 12, or 15.

5. The compound of claim 1, wherein said “q” and “o” in said enzymatic nucleic acid molecule are of the same length.

6. The compound of claim 1, wherein said “q” and “o” in said enzymatic nucleic acid molecule are of different length.

7. The compound of claim 1, wherein said chemical linkages in the enzymatic nucleic acid molecule are phosphate ester, amide, phosphorothioate, or phosphorodithioate linkages.

8. The compound of claim 1, wherein said C in the enzymatic nucleic acid molecule is 2′-deoxy-2′-NH₂ or 2′-deoxy-2′-O-NH₂.

9. The compound of claim 1, wherein said enzymatic nucleic acid molecule is chemically synthesized.

10. The method of claim 1, wherein said enzymatic nucleic acid molecule comprises at least one ribonucleotide.

11. The compound of claim 1, wherein said enzymatic nucleic acid molecule comprises no ribonucleotide residues.

12. The compound of claim 1, wherein said enzymatic nucleic acid molecule comprises at least one 2′-amino modification.

13. The compound of claim 1, wherein said enzymatic nucleic acid molecule comprises at least three phosphorothioate modifications.

14. The compound of claim 13, wherein the phosphorothioate modification is at the 5′-end of said enzymatic nucleic acid molecule.

15. The compound of claim 1, wherein said enzymatic nucleic acid molecule comprises a 5′-cap,a 3′-cap, or both a 5′-cap and a 3′-cap.

16. The compound of claim 15, wherein said 5 ′-cap is a phosphorothioate modification.

17. The compound of claim 15, wherein said 3′-cap is an inverted abasic moiety.

18. IThe compound of claim 1, wherein Z′ and Z comprise G and C respectively.

19. The compound of claim 1, wherein W comprises 5′-GAAA-3′.

20. The compound of claim 1, wherein W comprises 5′-UUAA-3′, 5′-CCGG-3′, 5′-AUAA-3′, 5′-GUAA-3′, 5′-GAUC-3′, 5′-UCGA-3′, or 5′-UAUA-3′.
